(19) United States
(12) Patent Application Publication
(10) Pub. No.: US 2005/0118142 A1
(43) Pub. Date: Jun. 2, 2005

(54) Cellular compositions which facilitate engraftment of hematopoietic stem cells while minimizing the risk of gvhd

(76) Inventor: Suzanne T Ildstad, Louisville, KY (US)

Correspondence Address: FROST BROWN TODD, LLC, 2200 PNC CENTER, 201 E. FIFTH STREET, CINCINNATI, OH, 45202, US

(21) Appl. No.: 10/485,842

(22) PCT Filed: Aug. 1, 2002
(86) PCT No.: PCT/US02/24402

(30) Foreign Application Priority Data
Aug. 1, 2001 (US) 60309243

(51) Int. Cl. A61K 45/00; C12N 5/08

(52) U.S. Cl. 424/93.7; 435/372

(57) ABSTRACT

The present invention provides for cellular compositions which facilitate engraftment of hematopoietic stem cells from a syngeneic, allogeneic or xenogeneic donor. The cellular compositions of the invention facilitate engraftment while minimizing the risk of graft versus host disease in the graft recipient. According to a preferred embodiment of the invention, a cell composition is provided, which cell composition comprises hematopoietic stem cells, such as CD34⁺ cells and/or facilitating cells, in combination with αβ TCR⁺ T cells. The invention also relates to methods of using the cellular compositions of the invention to induce donor specific tolerance in a recipient, thus allowing the transplantation of donor organs, cells and tissues. Also disclosed are methods of treating leukemia and cancer as well as infectious diseases caused by viruses.

### 1. FIELD OF THE INVENTION

The invention relates generally to the field of hematopoietic progenitor or stem cell transplantation. In particular, it relates to the isolation, characterization and uses of different cell populations which facilitate the engraftment of hematopoietic progenitor cells into a recipient from a syngeneic, allogeneic or xenogeneic donor. According to a preferred embodiment of the invention, a cell composition is provided, which cell composition comprises hematopoietic progenitor cells, such as CD34⁺ cells, in combination with αβ TCR⁺ T cells. Preferably, the composition also contains facilitating cells. Co-administration of hematopoietic progenitor cells with various cell populations found in either bone marrow or peripheral blood within specified ranges maximizes the potential of engraftment while minimizing the risk of incurring graft versus host disease (GVHD) in the recipient. Facilitation of engraftment of hematopoietic stem cells results in lymphohematopoietic chimerism thus permitting the permanent acceptance of donor cells, tissues and organs. Therefore the cellular compositions and methods of the invention will have a wide range of applications, including but not limited to, lympho-hematopoietic immune reconstitution by bone marrow and/or hematopoietic progenitor cell transplantation for the treatment of cancers, leukemias, anemias, autoimmunity, immunodeficiency, viral infections and metabolic disorders as well as facilitation of solid organ, tissue and cellular transplantation.

### 2. BACKGROUND OF THE INVENTION

The goal of hematopoietic progenitor cell or stem cell transplantation is to achieve the successful engraftment of donor cells within a recipient host such that immune and/or hematopoietic chimerism results. Chimerism is the reconstitution of the various compartments of the recipient's hematoimmune system with donor cell populations bearing major histocompatability complex (MHC) molecules derived from both the allogeneic or xenogeneic donor and a cell population derived from the recipient or alternatively the recipient's hematoimmune system compartments which can be reconstituted with a cell population bearing MHC molecules derived from only the allogeneic or xenogeneic marrow donor. Chimerism may vary from 100% (total replacement by allogenic or xenogeneic cells) to low levels detectable only by molecular methods. Chimerism levels may vary over time and be permanent or temporary.

Hematopoietic progenitor cells or stem cells are pluripotent cells that are capable of reconstituting a recipient's hematoinmmune system. Early hematopoietic progenitor cells are characterized by the presence of several cell surface markers, including CD34 (see, e.g., European patent application EP 0 451 611 A2). Reconstitution of the recipient's hematoimmune system is accomplished by transferring a heterogeneous population of cells, including hematopoietic stem or progenitor cells, derived from the donor's bone marrow or peripheral blood to the graft recipient. The challenge to achieving successful donor cell chimerism involves balancing the outcomes of graft rejection, graft versus leukemia (when the graft is performed in the context of treating a patient suffering from leukemia), immune reconstitution and graft versus host disease (GVHD). Graft rejection occurs when the donor cells fail to reconstitute the target compartments of the recipient's hematoimmune system. GVHD occurs when the graft of the donor cells is successful, but the immunocompetent donor cells recognize and attack the recipient's organs and tissues. Graft versus leukemia (GVL) is the recognition and destruction of residual leukemia cells by transplanted immune cells. When the aforementioned circumstances are optimally achieved, the immunological circumstance is generally associated with tolerance.

Bone marrow and/or stem cell transplantation has applications in a wide variety of clinical settings, including solid organ transplantation. A major goal in solid organ transplantation is the engraftment of the donor organ without a graft rejection immune response generated by the recipient, while preserving the immunocompetence of the recipient against other foreign antigens. Typically, nonspecific immunosuppressive agents such as cyclosporine, methotrexate, steroids and FK506 are used to prevent host rejection responses. They must be administered on a daily basis and if stopped, graft rejection usually results. However, nonspecific immunosuppressive agents function by suppressing all aspects of the immune response, thereby greatly increasing a recipient's susceptibility to infections and diseases, including cancer. Furthermore, despite the use of immunosuppressive agents, graft rejection still remains a major source of morbidity and mortality in human organ transplantation. It would therefore be a major advance if tolerance can be induced in the recipient.

The only known clinical circumstance in which complete systemic donor-specific transplantation tolerance has been induced is allogeneic hematopoietic stem cell transplantation (HSCT). (See Qin et al., 1989, J. Exp. Med. 169:779; Sykes et al., 1988, Immunol. Today 9:23; Sharabi et al., 1989, J. Exp. Med. 169:493). This has been achieved in neonatal and adult animal models as well as in humans by total lymphoid irradiation, total body irradiation or immunosuppressive chemotherapy of a recipient followed by bone marrow transplantation with donor cells. The success rate of HSCT is, in part, dependent on the ability to closely match the MHC of the donor cells with that of the recipient cells. The MHC is a gene complex that encodes a large array of glycoproteins expressed on the surface of both donor and host cells that are vital to normal function of the immune system, but are also the major targets of the transplantation rejection immune response. In humans, MHC is referred to as Human Leukocyte Antigen (HLA). HLA genes are inherited in a Mendelian fashion, hence, the only hope for a donor with an identical set of HLA proteins is in a sibling with the identical inheritance pattern. Transplants from a matched sibling donor are still associated with significant levels of GVHD, but meet with a high degree of success. However, when allogeneic bone marrow transplantation is performed between two MHC-mismatched individuals of the same species, common complications involve failure of engraftment, poor immunocompetence and a high incidence of GVHD.

GVHD is a potentially lethal complication in bone marrow transplantation, which occurs in about 35-50% of recipients of untreated HLA-identical marrow grafts (Martin et al., 1985, Blood 66:664) and up to 80% of recipients of HLA-mismatched marrow. Unfortunately, only 30% of patients generally have a suitably matched HLA-identical family member donor, and thus most patients are either excluded from being considered for bone marrow transplantation, or if they are transplanted, must tolerate a high risk of GVHD. GVHD results from the ability of immunocompetent mature immune cells, largely αβ TCR⁺ T cells, in the donor graft to recognize host tissue antigens as foreign and produce an adverse immunologic reaction.

Recent studies in bone marrow transplantation suggest that the major cause of GVHD is T cells, as the removal of T cells from the donor cell preparation was associated with a reduction in the incidence of GVHD. (Vallera et al., 1989, Transplant, 47:751; Rayfield, 1984, Eur. J. Immunol., 4:308; Vallera, 1982, J. Immunol., 128:871; Martin and Korngold, 1978, J. Exp. Med., 148(6):1687; Prentice, 1984, Lancet 1(8375): 472). After T cells were implicated as the predominant mediator of GVHD in animal models, aggressive protocols for T cell depletion (TCD) of human donor bone marrow were instituted. Technology now exists to deplete bone marrow of T cells. These techniques include the use of monoclonal antibodies (in conjunction with magnetic beads, immunotoxins or complement lysis), gradient fractionation, and soybean lectin agglutination. (See Kernan, 1994, Bone Marrow Transplantation, Oxford: Blackwell Scientific Publications, p. 124-35).

Although TCD decreased the incidence of GVHD dramatically, TCD was accompanied by a significant increase in the failure of engraftment, indicating that T cells might also play a facilitating role in bone marrow engraftment. (Soderling, J. Immunol., 1985, 135:941; Vallera, 1982, Transplant. 33:243; Pierce, 1989, Transplant., 48(2):289). TCD in the context of treating leukemia patients is also associated with an increased risk of leukemia relapse. It is therefore believed that T cells contained in the donor graft are instrumental in mediating this anti-leukemic effect known as GVL (Champlin et al. 1996, Acta Haematol 95: 157). In addition, the infused T cells probably produced immunologic effects against viruses and other pathogens, as evidenced by the increase in opportunistic infections in patients after TCD transplants.

The increase in failure of engraftment in human recipients ranges from about 5-70% of total patients and is related to the degree of MHC disparity between the donor and recipient and the breadth and completeness of TCD (Blazer, 1987, UCLA Symp., p. 382; Filipovich, 1987, Transplant., 44(1):62; Martin et al., 1985, Blood 66:664; Martin et al., 1988, Adv. Immunol. 40:379). Patients with failed engraftment usually die even if a second bone marrow transplant is performed. Consequently, most transplant institutions in the United States have abandoned TCD of donor bone marrow and, thus, must tolerate a high level of GVHD, which leads to significant morbidity and mortality. Thus, the application of bone marrow transplantation as a form of treatment is limited only to settings where the potential of GVHD is clearly outweighed by the potential benefit.

The implication that T cells might participate in both harmful GVHD reactions and helpful outcomes such as engraftment facilitation, GVL and hematoimmune reconstitution was an enigma that existed for a long time in the scientific community. Investigators began to search for the possible existence of a bone marrow component that could facilitate bone marrow engraftment but was removed during TCD. Identification and purification of this facilitating component would potentially allow the design of transplant protocols to selectively prevent GVHD, while preserving the cells that can enhance engraftment and thus allow the application of allogeneic and xenogeneic bone marrow transplantation to be used in a wide variety of clinical settings.

Cell populations that mediate graft facilitating or anti-leukemic effects have been identified in murine models (See U.S. Pat. No. 5,772,994; Kaufman et al., Thirty Fifth Annual Meeting of the American Society of Hematology, St. Louis, Mo., 1993, 82(10 Suppl. 1):456A). 76 TCR⁺ T cells have been shown to possess anti-leukemic activity against ALL (Lamb et al., 2001, Bone Marrow Transplantation 27:601). NK cells mismatched at receptor loci have also been shown to possess anti-leukemic activity (Valiante et al., 1997, Biol. Blood Marrow Transplant 3(5):229). αβ TCR⁺ T cells have been shown to possess suppressive activity against a wide variety of leukemias and other malignancies. Megadoses (>10⁷ kg/Ideal Body Weight) of CD34⁺ hematopoietic stem cells have been shown to have a beneficial effect in achieving engraftment across MHC barriers with little GVHD in some leukemia patients (Reisner et al.,1999, Ann. N.Y. Acad. Sci. 872:336). A subset of CD34⁺ cells called “veto cells” may induce anti-host unresponsiveness in the infused T cells (Gur et al., 1999, Blood 94(Suppl.):391; Bachar-Lustig et al., 1999, Blood 94:3212). Conversely, doses of 10⁵ cells/kg recipient body weight using cord blood have also produced engraftment (Laughlin et al., 2001, N. Eng. J Med 3(5):229). The chimeric immune system must also function to recognize and destroy pathogens.

It has also been reported that a distinct cell type bearing cell surface markers including CD8⁺, CD3⁺, Thy1⁺, ClassII^{dim/intermediate}, CD45⁺, αβ TCR⁻ T cells, and γδ TCR⁻ T cells can facilitate allogeneic and xenogeneic engraftment with little GVHD (U.S. Pat. No. 5,772,994; Kaufman et al., supra). In addition, studies have suggested that T cells, generally, may play a role in facilitating engraftment (Lapidot et al., 1992, Blood, 80(9):2406; Kernan et al.,1986, Blood, 68(3):770). These studies have not defined specific T cell subsets or doses that may be beneficial in facilitating engraftment. Studies have been done, using a mouse model, which suggest that αβ TCR⁺ T cells and γδ TCR⁺ T cells may facilitate engraftment (Drobyski et al., 1997, Blood, 89(3):1100). However, the predicted beneficial dose ranges of the T cell subsets in this study are not within the ranges provided for by the instant invention.

The invention disclosed herein describes compositions of cells isolated from bone marrow and/or peripheral blood and the uses of these cellular compositions to facilitate desired clinical outcomes including bone marrow and/or hematopoietic progenitor cell engraftment by minimizing the risk of both graft rejection and GVHD, while allowing for hematopoietic and/or immune reconstitution. In the context of treating leukemia, the cellular compositions of the invention maintain the graft versus leukemia effect.

### 3. SUMMARY OF THE INVENTION

The present invention provides cellular compositions and methods of treating a host mammal in need of a hematopoietic progenitor cell transplant by identifying cellular populations that facilitate achieving desirable outcomes such as engraftment of hematopoietic stem cells. According to a preferred embodiment of the invention, a cell composition is provided, which cell composition comprises hematopoietic progenitor cells, such as CD34⁺ cells, in combination with αβ TCR⁺ T cells. The cells utilized in the cellular compositions of the invention may be derived from any physiological source, for example, but not as a limitation, bone marrow.

As noted in Section 2, supra, hematopoietic progenitor cells carrying the CD34 marker, when administered in large numbers, appear to correlate with a reduced time to engraftment (Reisner et al., supra). Conversely, the use of low numbers of CD34⁺ cells correlates with much longer times to engraftment. However, obtaining large numbers of CD34⁺ early progenitor cells can be difficult. The present invention allows for the use of smaller quantities of CD34⁺ cells in allogeneic hematopoietic stem cell transplants by combining the hematopoietic progenitor cells with certain populations of cells that facilitate engraftment. Concentrations of CD34⁺ cells according to the compositions and methods of this invention can be in the range of about 0.5×10⁶ to about 2.5×10⁶ cells/kg ideal body weight (IBW), preferably in the range of about 0.9×10⁶ to about 2.0×10⁶ cells/kg ideal body weight (COW).

More specifically, applicants have discovered that T cells that carry the of TCR marker (referred to herein as αβ TCR⁺ T cells) are correlated with producing a shorter time to engraftment or both neutrophils and platelets when they are present in bone marrow transplants. As exemplified infra in Section 6.12, this subset of T cells correlates with producing shorter times to engraftment using a univariate and/or multivariate mathematical analysis. Thus, in one embodiment, the invention comprises a cellular composition comprising CD34⁺ hematopoietic cells and αβ TCR⁺ T cells. T cells that carry the γδ TCR marker (referred to herein as γδ TCR⁺ T cells) also show a tendency to produce shorter times to engraftment of both platelets and neutrophils and they are not correlated with producing acute GVHD. Accordingly, an alternative embodiment of the present invention comprises a cellular composition comprising CD34⁺ hematopoietic cells, αβ TCR⁺ T cells, and γδ TCR⁺ T cells. Another embodiment of the present invention comprises a cellular composition comprising CD34⁺ hematopoietic cells, facilitating cells, αβ TCR⁺ T cells, and γδ TCR⁺ T cells. In addition, since NK cells correlate with producing shorter times to engraftment in a univariate statistical model (See Section 6.12 infra), still another embodiment of the invention comprises any of the cellular compositions described above in combination with NK cells.

Finally, B cells do not appear to facilitate engraftment and are a source of post-transplant complication, particularly post-transplant lymphoproliferative disorder. Therefore, according to one embodiment of the invention, the number of B cells in hematopoietic stem cell transplantation is limited.

The invention therefore relates to cellular compositions comprising CD34⁺ cells in combination with one or more of the following cell populations: αβ TCR⁺ T cells, γδ TCR⁺ T cells, NK cells and/or B cells in appropriate concentrations such that host engraftment is facilitated while the risk of inducing GVHD and/or other post-transplant complications is minimized. Preferably, the cellular compositions comprising CD34⁺ cells in combination with facilitating cells along with one or more of the following cell populations: αβ TCR⁺ T cells, γδ TCR⁺ T cells, NK cells and/or B cells in appropriate concentrations such that host engraftment is facilitated while the risk of inducing GVHD and/or other post-transplant complications is minimized.

In promoting hematopoietic engraftment in a host that may be HLA-mismatched, including, for example, allogeneic, or xenogeneic transplants, the invention may be used for the generation of donor tolerance by the host. Coexistence of donor and host marrow or complete replacement of host marrow by donor cells and the subsequent creation of a mammalian chimera by the invention provides a new method for generating tolerance to either solid organ or tissue transplants. Transplantation of donor hematopoietic progenitor cells allows the subsequent and/or simultaneous transplantation of solid organs or tissues from the same donor such as but not limited to heart, kidney and liver. The recipients' new immune system recognizes the transplanted organs as ‘self’ as opposed to ‘non-self.’

Thus, the invention provides methods for treating a host mammal with an allogeneic or xenogeneic hematopoietic progenitor cell preparation to allow engraftment of the new cell material into the host. The host mammal usually undergoes immunosuppression before administration of the transplant of hematopoietic cells. Immunosuppression of the host reduces its ability to recognize and reject the donor cells of the graft. Pharmacologic methods, immunologic methods and/or irradiation may achieve this immunosuppression of the host, referred to herein as conditioning, for example. By utilizing the cell compositions and methods of the invention, the clinical outcome of the recipient prepared by various conditioning approaches can be enhanced. The clinical outcomes or endpoints that can be influenced include but are not limited to: (i) serious acute graft versus host disease (defined as grades 3 and 4), (ii) chronic graft versus host disease, (iii) post-transplant lymphoproliferative disease, (iv) engraftment of both platelets and neutrophils, (v) immune reconstitution, (vi) disease relapse (examples include leukemia, lymphoma and sickle cell disease), (vii) overall survival or (viii) tolerance.

The compositions and methods of this invention can be used in the treatment of any disease or condition requiring a hematopoietic progenitor or stem cell transplant. By way of example, but not as a limitation, such diseases can include: cancer, leukemia, lymphoma, or any malignancy of hematolymphoid origin, autoimmune disease, AIDS, any disease resulting in immunodeficiency, sickle cell disease, anemia, diabetes or any viral infection. The compositions may also be used to treat a recipient in need of an organ or tissue transplant. Non-limiting examples of organ transplants for which the compositions might be used include heart, lung, liver, kidney, pancreas or skin graft. The compositions may also be used in tissue transplants such as islet cells of the pancreas or dopamine-producing brain cells. The examples provided are not intended to be limiting and one skilled in the art would appreciate that the invention would have application in any clinical setting where transplantation is used.

### 3.1. Definitions

As used herein, “recipient” means any mammal, including humans.

As used herein, “donor” means any mammal, including humans.

As used herein, “chimera” means a recipient comprising cells from the recipient and cells from at least one donor.

As used herein, “syngeneic” means of donor origin wherein the donor is genetically identical to the recipient.

As used herein, “allogeneic” means of donor origin wherein the donor is of the same species as the recipient.

As used herein, “xenogeneic” means of donor origin wherein the donor is of a different species than the recipient.

As used herein “autologous” means cells of the recipient individual which remain or have been removed and reinfused.

As used herein, “mixed chimeric hematoimmune system” means a recipient hematopoietic and/or immune system comprising from about molecularly detectable levels to about 99% allogeneic or xenogeneic cells and the remaining percentage of autologous and/or syngeneic cells.

As used herein, “completely allogeneic chimeric hematoimmune system” means a recipient hematopoietic and/or immune system created through the administration of both allogeneic and syngeneic and/or autologous cells and comprising virtually 100% allogeneic cells but in which some residual syngeneic and/or autologous cells providing for a limited number of immunological cell lineages may exist.

As used herein, “completely xenogeneic chimeric hematoimmune system” means a recipient hematopoietic and/or immune system created through the administration of both syngeneic and/or autologous and xenogeneic cells and comprising virtually 100% xenogeneic cells but in which some residual syngeneic and/or autologous cells providing for a limited number of immunological cell lineages may exist.

As used herein αβ TCR⁺ T cells means cells which express the αβ T cell receptor (TCR) on the cell surface and do not express the γδ TCR on the cell surface.

As used herein TCR⁺ T cells means cells which express the γδ TCR on the cell surface and do not express the 0 TCR on the cell surface.

As used herein hematopoietic progenitor or stem cells mean progenitor cells capable of, upon transplantation, reconstituting a recipient's hematoimmune system and bearing the surface marker CD34.

As used herein, “hematopoietic facilitatory cells” (facilitating cells or FC) are cells from bone marrow or other physiological sources of hematopoietic cells that generally have a phenotype of CD8⁺, αβ TCR−, and γδ TCR−. Preferably, the hematopoietic facilitatory cells have a phenotype of CD3⁺, CD8⁺, αβ TCR⁻ and γδ TCR⁻ as determined by antibody staining and flow cytometry, which hematopoietic facilitatory cells are capable of facilitating engraftment of bone marrow cells.

Furthermore, the FC are generally characterized by being αβ-TCR⁻, γδ-TCR⁻, CD4⁻, CD5⁻, CD16⁻, CD19⁻, CD20⁻, CD56⁻, mature myeloid lineage (CD14), Class II⁺, CD45⁺, CD45R⁺, THY1⁺, CD8⁺, and CD3⁺. A high concentration of FC may be obtained by positive separation of a mixture of hematopoietic cells into a facilitatory cell containing fraction which is Class II⁺ and THY1⁺. The Class II+ fraction may be further separated based on staining intensity and the Class II bright population eliminated.

The facilitatory cells express a unique profile of cell surface markers: Thy-1⁺, CD3⁺, CD8⁺, CD45⁺ CD45R⁺, MHC class II⁺, CD4⁻, CD5⁻, CD14⁻, CD16⁻, CD19⁻, CD20⁻, CD56⁻, γδ-TCR⁻ and αβ-TCR⁻. Although the Applicant's own work supports the CD3⁺ phenotypic characterization of the hematopoietic facilitatory cell population, recent work of other groups raises the possibility that these cells may, in fact, be CD3⁻.

As used herein Ideal Body Weight (IBW) is defined and calculated as follows:
Ideal Body Weight (IBW) (>60 inches in height)
Male: 50.0 kg+(2.3 kg×number of inches over 60 inches)
Female: 45.5 kg+(2.3 kg×number of inches over 60 inches)

For patients less than 60 inches tall, the EBW is determined by locating the individual's height on a gender-appropriate growth chart, then locating the 50^{th} percentile weight for height on the growth chart.

Because this is calculated according to height and sex it will not fluctuate.

### 4. BRIEF DESCRIPTION OF THE FIGURES

FIG. 1 The major differences in processes 1, 2, 3, and 4 used to process bone marrow for transplantation are illustrated by flowchart. Boxes indicate changes in the methods used to process bone marrow.

FIG. 2 Kaplan Meier plot showing the probability of neutrophil engraftment as a function of days since bone marrow transplant. Patients were divided into 2 groups: those that received a CD34⁺ cell dose of>0.9×10⁶ cells/kg EBW and those that received a CD34⁺ cell dose<0.9×10⁶ cells/kg IBW.

FIG. 3 Kaplan Meier plot showing the probability of neutrophil engraftment as a function of days since bone marrow transplant. Patients were divided into 2 groups: those that received a CD34⁺ cell dose of>1.9×10⁶ cells/kg IBW and those that received a CD34⁺ cell dose<1.9×10⁶ cells/kg IBW.

FIG. 4 Kaplan Meier plot showing the probability of platelet engraftment as a function of days since bone marrow transplant. Patients were divided into 2 groups: those that received a CD34⁺ cell dose of>0.9×10⁶ cells/kg IBW and those that received a CD34⁺ cell dose<0.9×10⁶ cells/kg IBW.

FIG. 5 Kaplan Meier plot showing the probability of platelet engraftment as a function of days since bone marrow transplant. Patients were divided into 2 groups: those that received a CD34⁺ cell dose of>1.9×10⁶ cells/kg IBW and those that received a CD34⁺ cell dose<1.9×10⁶ cells/kg IBW.

FIG. 6 The median difference in days to neutrophil engraftment based upon αβ T cell dose cutoff.

FIG. 7 Box plot showing the incidence of severe acute GVHD (aGVHD) based upon the αβ T cell dose in the graft.

FIG. 8 Recovery of various cell populations using a COBE Spectra™ processing step versus a percoll gradient step. Shown is the comparison of CD34⁺ and lymphoid cell recoveries between the two methods. The figure shows the recovery of CD34⁺ and lymphocyte subsets is higher using the COBE Spectra™ process compared to the Percoll process.

### 5. DETAILED DESCRIPTION OF THE INVENTION

The invention relates to the field of hematopoietic cell transplantation. The invention provides for specific cellular compositions that contain both donor hematopoietic progenitor cells as well as specific populations of donor cells that facilitate the engraftment of the donor hematopoietic cells into a recipient host, while minimizing the risk of GVHD. According to a preferred embodiment of the invention, a cell composition is provided, which cell composition comprises hematopoietic progenitor cells, such as CD34⁺ cells, in combination with αβ TCR⁺ T cells. Another embodiment of the present invention comprises a cellular composition comprising CD34⁺ hematopoietic cells, facilitating cells, αβ TCR⁺ T cells, and γδ TCR⁺ T cells. The donor and recipient may be allogeneic, syngeneic, or xenogeneic. The donor hematopoietic cells can be derived from bone marrow, peripheral blood, cord blood, liver spleen or any organ within which the cellular populations, which facilitate engraftment, reside. Thus, the invention also provides for methods to isolate, enrich for or purify the cellular populations, which produce and/or facilitate hematopoietic progenitor cell engraftment.

The invention also encompasses the use of cellular compositions of the invention in the treatment of leukemia or cancer, autoimmune diseases, anemia, sickle cell disease and infectious diseases, including, but not limited to viral infections. By way of example, but not as a limitation, such infectious diseases can include HIV, hepatitis C, and hepatitis B. The invention also provides for cellular compositions which can be used in the field of solid organ, cell, or tissue transplantation. The invention also relates to the use of cellular compositions, which facilitate the engraftment of donor hematopoietic cells into a recipient host, wherein the host is in need of a solid organ, tissue or cellular transplantation. By way of example, but not as a limitation, the cellular compositions of the invention can be used in the transplant of heart, lung liver, kidney, islet cells, skin, endocrine organs, or pancreas.

The invention is discussed in more detail in the subsections below, solely for purposes of description and not by way of limitation. For clarity of discussion, the specific procedures and methods described herein are exemplified using a human model; they are merely illustrative for the practice of the invention. Analogous procedures and techniques are equally applicable to all mammalian species, including non-human subjects, in terms of deriving the cell compositions used as donor and a human recipient receiving such cells in transplantation. Therefore, human or non-human cell compositions having a similar phenotype and function may be used under the conditions described herein.

### 5.1. Characterization of Cell Compositions of the Invention

According to the present invention, allogeneic or xenogeneic (donor) hematopoietic progenitor cells are administered following conditioning, in combination with certain cell populations that affect desired clinical endpoints, such as GVHD and engraftment, in a dose dependent manner. More particularly, the present invention indicates that certain ranges of these cell populations present in the donor graft facilitate engraftment of donor marrow and/or progenitor cells in a recipient host. By setting upper and lower limits for transplantation on the number of these engraftment-facilitating cells (i.e., cells/kg body weight of the recipient host) or the absolute number of these cells, engraftment of donor hematopoietic progenitor cells can be facilitated while minimizing the risks of GVHD.

The present invention thus provides for specific compositions comprising CD34⁺ hematopoietic progenitor cells, in readily obtained quantities, in combination with one or more of the following cellular populations: ce TCR⁺ T cells, γδ TCR⁺ T cells, NK cells or B cells. Another embodiment of the present invention comprises a cellular composition comprising CD34⁺ hematopoietic cells, facilitating cells, αβ TCR⁺ T cells, and γδ TCR⁺ T cells. The present invention also provides for specific compositions comprising facilitating cells, in readily obtained quantities, in combination with one or more of the following cellular populations: αβ TCR⁺ T cells, γδ TCR⁺ T cells, NK cells or B cells. In yet another embodiment, the composition additionally comprises CD34⁺ cells.

When administered at appropriate concentrations to a patient in need of a hematopoietic progenitor cell transplant, these cells can facilitate engraftment and/or other positive endpoints, while minimizing the risk of GVHD. The present invention also provides methods of preparing the cellular compositions of the invention, which compositions comprise CD34⁺ cells, in combination with one or more of the following cellular populations: αβ TCR⁺ T cells, γδ TCR⁺ T cells, NK cells or B cells, and which compositions facilitate engraftment of hematopoietic progenitor cells from a donor into an allogeneic, syngeneic or xenogeneic host or recipient in need of a hematopoietic progenitor cell transplant. The present invention also provides methods of preparing the cellular compositions of the invention, which compositions comprise facilitating cells, in combination with one or more of the following cellular populations: αβ TCR⁺ T cells, γδ TCR⁺ T cells, NK cells or B cells, and which compositions facilitate engraftment of hematopoietic progenitor cells from a donor into an allogeneic, syngeneic or xenogeneic host or recipient in need of a hematopoietic progenitor cell transplant. In yet another embodiment, the composition additionally comprises CD34⁺ cells.

According to one embodiment of the invention, generally at least about 0.5×10⁶ cells/kg IBW, preferably at about 1×10⁶ cells/kg IBW, and more preferably at about 1×10⁶ cells/kg IBW of the CD34⁺ hematopoietic progenitor cells are used.

Preferably, the CD34⁺ hematopoietic progenitor cells are combined with αβ TCR⁺ T cells at a dose of preferably from about 0.1 to about 3.0×10⁵ cells/kg IBW, to form the cellular compositions of the invention for administration to a recipient host.

According to a preferred embodiment of the invention, CD34⁺ hematopoietic progenitor cells, generally at least about 0.5×10⁶ cells/kg IBW, preferably at about 1×10⁶ cells/kg IBW, and more preferably at about 1×10⁶ cells/kg IBW are combined with αβ TCR⁺ T cells at a dose of preferably about 0.1 to about 3.0×10⁵ cells/kg IBW, to form the cellular compositions of the invention for administration to a recipient host. In an alternative embodiment of the invention, γδ TCR⁺ T cells can be added to the above cellular compositions, for example, at a cell dose of at least about 1×10⁵ cells/kg IBW, preferably at least about 1.5×10⁵ cells/kg IBW, resulting in a cellular composition comprising CD34⁺ hematopoietic cells, αβ TCR⁺ T cells as well as γδ TCR⁺ T cells. According to one embodiment of the invention, γδ TCR⁺ T cells are added at a cell dose of about 7×10⁶ cells/kg IBW. In still another embodiment, NK cells can be added to any one of the above cellular compositions, for example, at a cell dose of about 0.1-2×10⁶ cells/kg IBW.

According to another embodiment of the invention, CD34⁺ hematopoietic progenitor cells, generally at least about 0.5×10⁶ cells/kg IBW, preferably at about 1×10⁶ cells/kg EBW, and more preferably at about 1×10⁶ cells/kg IBW are combined with αβ TCR⁺ T cells at a dose of preferably about 0.1 to about 3.0×10⁵ cells/kg IBW, and with facilitating cells, generally at least about 0.004×10⁶ cells/kg IBW, preferably at about 0.05×10⁶ cells/kg IBW, and more preferably at about 0.5×10⁶ cells/kg IBW to form the cellular compositions of the invention for administration to a recipient host. Generally, the facilitating cells will be present in at a concentration of no greater than about 2×10⁶ cells/kg IBW

In another embodiment the invention encompasses a cell composition comprising CD34⁺ cells at a cell dose of about 21.9×10⁶ cells/kg IBW, and αβ TCR⁺ T cells in the range of about 0.4-1.25×10⁵ cells/kg IBW. γδ TCR⁺ T cells may be added to this cell composition at about>1.5×10⁵ cells/kg IBW. Optionally, NK cells can also be added in the range of about 0.1-2.0×10⁶ cells/kg IBW.

A preferred embodiment of the invention encompasses a cell composition comprising CD34⁺ cells at a cell dose of at least about 2×10⁶ cells/kg IBW, and αβ TCR⁺ T cells in the range of from about 0.5 to about 3.0×10⁵ cells/kg IBW. γδ TCR⁺ T cells and/or NK cells may be optionally added.

According to yet another embodiment, the B cell dose of the composition should not exceed 1×10⁵ cells/kg IBW.

### 5.2. Isolation of Cellular Compositions of the Invention

The present invention provides for methods of enriching and/or purifying cellular populations, which facilitate engraftment from bone marrow or other physiological sources of hematopoietic progenitor cells. The activity of these cell populations allows for their use within a specified range such that successful engraftment of a donor transplant as measured by neutrophil count and/or platelet count is achieved while the risk of GVHD is minimized. The cellular populations that facilitate engraftment may be isolated from any tissue in which they reside, for example but not as a limitation, from bone marrow, using a variety of separation procedures, including separations based on the presence or absence of specific cell surface molecules or markers. The present invention also provides for methods of culturing and expanding, in vitro or in vivo in the donor prior to collection, cell populations which facilitate engraftment of hematopoietic progenitor cells and other desired clinical endpoints.

According to one embodiment, the invention comprises methods of isolating hematopoietic progenitor cells and/or cell populations that facilitate engraftment. Bone marrow is harvested by standard aspiration techniques with a goal of obtaining, e.g., 3×10⁸ total nucleated cells/kg. After an initial erythrocyte, plasma and platelet removal step, using the COBE 2991™ (COBE BLT, Inc., Lakewood, Colo.) according to the manufacturer's instructions, step gradient centrifugation is performed, e.g., using a gradient comprised of CELL/FLEX 10530 (Atlanta Biologicals, Norcross, Ga.) and CELL/FLEX 1077®, to remove red blood cells, granulocytes, platelets and cell debris. The cells are centrifuged through the step gradient at 1400×g for 25 minutes. The cells are resuspended in DPBS (Dulbeco's Phosphate Buffered Saline)+10 mg/ml HSA (human serum albumin). In a preferred embodiment, mononuclear cells from the bone marrow harvest are separated from red blood cells, platelets, granulocytes and cell debris using the automated COBE Spectra™ Apheresis system (COBE BLT, Inc. Lakewood, Colo.).

After the mononuclear cells have been isolated from the harvested bone marrow, those cells are subjected to antibody depletion, e.g., using a monoclonal antibody (MAb) against the αβ TCR. After antibody binding, the antibody-bound cells are depleted using magnetic separation techniques, e.g., ferromagnetic beads and a MAXSEP™ Magnetic Cell Separation System (Baxter Healthcare Corp., Biotech Group, Immunotherapy Division, Deerfield, Ill.). The unbound cells are harvested and preferably washed and subjected to a second antibody depletion step, e.g., MAbs directed against αβ TCR, CD56 and CD19, followed by a second round of magnetic depletion using ferromagnetic beads and the MAXSEP™ system. The unbound cells are harvested and used for bone marrow transplants. Cells recovered in the wash steps are used to enrich the final product with the desired concentrations of cell populations determined to facilitate engraftment while minimizing the risk of GVHD, such as αβ TCR⁺ T cells. See Example Section 6, infra, for further details of the methods of the invention.

Although bone marrow is preferred, other physiologic sources of hematopoietic progenitor cells may be utilized, for example, the spleen, thymus, blood, cord blood, embryonic yolk sac, or fetal liver. Bone marrow may be removed from any bone cavity by various methods well known to those skilled in the art. Typically, the bone marrow is filtered, centrifuged and resuspended.

As noted above, once a source of hematopoietic progenitor cells is obtained, specific cellular populations, which facilitate desired clinical endpoints, such as engraftment, may be obtained by various methods. These techniques may include, as examples, but not as a limitation, the use of specific antibodies which preferably bind to specific markers on the cells to select those cells possessing or lacking various markers, flow cytometry using a fluorescence activated cell sorter (FACS) and specific fluorochromes, biotin-avidin and biotin-streptavidin separations using biotin conjugated to cell surface marker-specific antibodies and avidin or streptavidin bound to a solid support such as affinity column matrix or plastic surfaces, magnetic separations using antibody-coated magnetic beads, deletional separations such as antibody and complement or antibody bound to cytotoxins or radioactive isotopes.

Separation via antibodies for specific markers may be by negative or positive selection procedures. In negative separation, antibodies are used which are specific for markers present on undesired cells. Cells bound by the antibody may be removed or lysed and the remaining desired mixture retained. In positive separation, antibodies specific for markers present on the desired cells are used. Cells bound by the antibody are separated and retained. It will be understood that positive and negative separations may be used substantially simultaneously or in a sequential manner or alone. It will also be understood that the present invention encompasses any separation technique that can isolate cells based on the characteristic phenotype or physical qualities of the cellular populations, which facilitate engraftment as, disclosed herein.

If desired, a separation may be initiated by techniques which can remove a large proportion of cell subsets, such as T cells, B cells, NK cells, dendritic cells and macrophages (MAC), as well as minor cell populations including megakaryocytes, mast cells, eosinophils, and basophils. Once removed, these cellular populations can be added back at a specified cell number or cell concentration such that a cellular composition that facilitates engraftment and minimizes the risk of GVHD is achieved.

Although separations based on specific markers are disclosed, it will be understood that the present invention encompasses any separation based on the characterization of the cellular compositions which facilitate desired clinical outcomes, such as engraftment, of donor hematopoietic cells disclosed herein, whether that separation is a negative separation, a positive separation, or a combination of negative and positive separations, and whether that separation uses cell sorting or some other technique, such as, for example, antibody plus complement treatment, column separations, panning, biotin-avidin technology, density gradient centrifugation, or other techniques known to those skilled in the art. It is understood that once various cell populations are removed from the composition they may be added back at appropriate concentrations such that the composition promotes desired clinical outcomes, e.g., facilitates engraftment, while minimizing the risk of GVHD. According to one embodiment of the invention, these cell populations can be added back after culture and expansion in vitro. It will be appreciated that the present invention encompasses these separations used on any mammal including, but not limited to humans, primates, baboons, rats, mice, and other rodents.

### 5.3. Uses of the Cell Compositions of the Invention

The cellular compositions of the invention enhance engraftment of bone marrow and/or hematopoietic progenitor donor cells in an allogeneic or xenogeneic recipient and thus they may be useful in facilitating various therapy protocols involving transplantation procedures. More specifically, formulation of a cellular composition comprising the appropriate concentrations or doses of engraftment-enhancing cellular populations, in combination with donor hematopoietic progenitor cells, provides a solution to the alternative problems of GVHD and failure of engraftment. For example, donor marrow containing hematopoietic progenitor or stem cells which has been depleted of T cells and reconstituted with appropriate engraftment-enhancing cell populations, including appropriate concentrations of αβ and/or γδ TCR⁺ T cells, may be used to improve transplantation outcomes.

The present invention thus provides for the use of the cellular compositions of the invention in establishing a mixed allogeneic or mixed xenogeneic chimeric hematoimmune system, completely allogeneic or completely xenogeneic chimeric hematoimmune system. If mixed or completely allogeneic or xenogeneic chimerism is desired, syngeneic or autologous cellular compositions that comprise cell populations that facilitate engraftment and progenitor cells may be administered along with the donor cell compositions. However, it is not required that engraftment-enhancing cell populations are used with other donor cells that are autologous or syngeneic to the host. Allogeneic or xenogeneic cell populations that facilitate engraftment may be used with MHC-matched bone marrow and/or hematopoietic progenitor cells to reconstitute a recipient, without co-administration of autologous or syngeneic donor cells. For allogeneic chimerism, donor and recipient are of the same species; for xenogeneic chimerism, donor and recipient are of different species.

In order to create such chimeric hematoimmune systems using the methods and compositions of this invention, a hematopoietic progenitor cell source such as bone marrow may be harvested from the long bones of the donor. According to an embodiment of the invention, a cellular composition comprising a high concentration of hematopoietic stem or progenitor cells such as CD34⁺ and/or facilitating cells is separated from the donor bone marrow as described in Section 5.2, supra, and as exemplified in Sections 6.6-6.9, infra. One or more cellular compositions comprising cell populations that facilitate engraftment, e.g., αβ TCR⁺ T cells, γδ TCR⁺ T cells and/or NK cells, are also separated from the donor bone marrow, also as described in Section 5.2, supra, and as exemplified in Sections 6.6-6.9, infra. The purified or enriched cell compositions are preferably mixed and then administered to the recipient. If these cellular compositions are separate compositions, they are preferably administered simultaneously, but may be administered separately within a relatively close period of time or a prescribed temporal sequence. The mode of administration is preferably but not limited to intravenous injection. Preferably the ex vivo time of the graft is minimized to provide an enhanced clinical outcome.

Once administered, it is believed that the cells home to various hematopoietic and immunopoietic sites in the recipient's body, including bone cavity, spleen, lymph node, fetal or adult liver, and thymus. The engrafted hematopoietic progenitor cells rapidly produce the terminally differentiated components of the blood such as erythrocytes, granulocytes and platelets. These cells mature in the bone marrow and require only nonspecific modifications by the reticuloendothelial system after leaving the bone marrow. Of the immune system cells, NK cells are most rapidly seen as products of engrafted marrow. Some progenitor cells require substantial processing and cell-cell interaction outside the bone marrow to become functional mature T and B cells. However, differentiated mature and naive cells infused in the donor cell aliquot can function very rapidly in the host.

Generally, in order to establish an allogeneic or xenogeneic chimeric hematoimmune system, the immune system of the recipient may be substantially destroyed prior to administration of the cell compositions of the invention. This may be accomplished by techniques known in the art. These techniques result in substantially full ablation of the bone marrow stem cells and immune cells of the recipient. However, there may be some resistant recipient stem cells and immune cells, which survive and continue to produce autologous blood immune cells. These techniques include, for example, lethally irradiating the recipient with selected levels of radiation, e.g., total body irradiation or total lymphoid irradiation, administering specific toxins or drugs to the recipient, administering specific monoclonal or polyclonal antibodies, in their native state or attached to toxins or radioactive isotopes or combinations of these techniques.

Alternatively, a recipient may be conditioned by immunosuppression and cytoreduction by the same techniques as are employed in substantially destroying a recipient's immune system, including, for example, irradiation, toxins, antibodies bound to toxins or radioactive isotopes, or some combination of these techniques. However, the level or amount of agents used is substantially smaller when immunosuppressing and cytoreducing than when substantially destroying the hematoimmune system. For example, substantially destroying a recipient's remaining lymphohematopoietic system often involves lethally irradiating the recipient with 950 rads (R) of total body irradiation (TBI). For humans, this involves lethally irradiating the recipient with 1200 cGy of total body irradiation (TBI). This level of radiation is fairly constant no matter the species of the recipient. Consistent xenogeneic (ratmouse) chimerism has been achieved with 750 R TBI and consistent allogeneic (mouse) chimeras with 600R TBI. Chimerism was determined by PBL typing and tolerance confirmed by mixed lymphocyte reactions (MLR) and cytotoxic lymphocyte (CTL) response.

As stated hereinbefore, the above disclosed methous may bc used for establishing both allogeneic chimerism and xenogeneic chimerism. Xenogeneic chimerism may be established when the donor and recipient as recited above are different species; for example, xenogeneic chimerism between rats and mice, between hamsters and mice, and between chimpanzees and baboons has been established. Xenogeneic chimerism between humans and other primates is also possible. Xenogeneic chimerism between humans and other mammals is equally viable.

It will be appreciated that, although the methods disclosed above involve one recipient and one donor, the present invention encompasses methods such as those disclosed in which progenitor cells and purified or enriched cells which facilitate engraftment from two donors are engrafted in a single recipient.

It will be appreciated that the present invention also provides methods of reestablishing a recipient's hematoimmune system by substantially destroying the recipient's immune system or immunosuppressing and cytoreducing the recipient's immune system, and then administering to the recipient syngeneic or autologous cell compositions comprising syngeneic or autologous purified or enriched cell populations that facilitate engraftment and hematopoietic progenitor cells which are MHC-identical to the cell populations which facilitate engraftment.

The ability to establish successful allogeneic or xenogeneic chimerism also allows for vastly improved survival of transplants of other tissues. The present invention provides for methods of transplanting a donor physiological component, such as, for example, organs, tissue, or cells. Examples of successful transplants in and between rats and mice using these methods include, for example, islet cells, skin, hearts, livers, thyroid glands, parathyroid glands, adrenal cortex, adrenal medullas, and thymus glands. The recipient's chimeric hematoimmune system is completely tolerant of the donor organ, tissue, or cells, but competently rejects third party grafts. Also, bone marrow and/or hematopoietic progenitor cell transplantation confers subsequent tolerance of organ, tissue, or cellular grafts which are genetically identical or closely matched to the bone marrow and/or hematopoietic progenitor cell previously engrafted.

Transplanted donor organ, tissue, or cells competently perform their physiologic function in the recipient. For example, transplanted islet cells function competently, and thereby provide an effective treatment for diabetes. In addition, transplantation of bone marrow and or hematopoietic progenitor cells using methods of the present invention can eliminate the autoimmune diabetic trait before insulin-dependence develops. Successful solid organ transplants between humans and animals may be performed using methods of the present invention involving hematopoietic stem cells and cell populations, which facilitate engraftment. For example, islet cells from other species may be transplanted into humans to treat diabetes in the human recipient after the disease is diagnosed or after the onset of insulin dependence. Major organs from animal donors such as, for example, pigs, cows or fish can solve the current problem of donor shortages. For example, 50% of patients who require a heart transplant die before a human donor is available. It has been demonstrated that permanent acceptance of endocrine tissue engrafts (thyroid, parathyroid, adrenal cortex, adrenal medulla, islets) occurs in xenogeneic chimeras after bone marrow transplantation from a genetically identical xenogeneic donor. Hence, mixed xenogeneic chimerism or fully xenogeneic chimerism established by methods of the present invention can be employed to treat autoimmune disorders, such as rheumatoid arthritis and lupus erythematosus.

The method of establishing a mixed or allogeneic chimeric hematopoietic and/or immunopoietic system may be performed before, during, or after the transplantation, but is preferably performed before the transplantation. The methods disclosed allow for both allotransplantation and xenotransplantation. Because the methods disclosed herein provide for donor-specific immunotolerance, many procedures previously necessary to resist rejection of the donor organ, tissue, or cells are unnecessary. For example, live bone and cartilage may be transplanted by the herein disclosed methods.

Cell expansion or cell farming technology can provide for a readily available supply of cell populations that facilitate engraftment, stem cells and genetically matched physiological donor components. For example, bone marrow and/or hematopoietic progenitor or engraftment-enhancing cells can be propagated in vitro in cultures and/or stored for future transplantation. According to one embodiment, these cells can be propagated through the use of growth factors such as GCSF. Cellular material from the same donor can be similarly stored for future use as grafts.

Beyond transplantation, the ability to establish a successful allogeneic or xenogeneic chimeric hematoimmune system can provide cures for various other diseases or disorders which are not currently treated by bone marrow transplantation because of the morbidity and mortality associated with GVHD. Autoimmune diseases involve attack of an organ or tissue by one's own immune system. In this disease, the immune system recognizes the organ or tissue as a foreign. However, when a chimeric hematoimmune system is established, the body relearns what is foreign and what is self. Establishing a chimeric immune system as disclosed can simply halt the autoimmune attack causing the condition. Also, autoimmune attack may be halted by reestablishing the victim's immune system after immunosuppression and cytoreduction or after immunodestruction with syngeneic or autologous cell compositions as described hereinbefore. Autoimmune diseases which may be treated by this method include, for example, type I diabetes, systemic lupus erythematosus, multiplesclerosis, rheumatoid arthritis, psoriasis, colitis, and even Alzheimer disease. The use of cell populations that facilitate engraftment plus stem cells can significantly expand the scope of diseases that can be treated using bone marrow transplantation.

Because a chimeric hematoimmune system includes hematopoietic progenitor cells from the donor hematoimmune system, deficiencies in the recipient hematoimmune system may be alleviated by a nondeficient donor hematoimmune system. Hemoglobinopathies such as sickle cell anemia, spherocytosis or thalassemia and metabolic disorders such as Hunters disease, Hurlers disease, and enzyme defects, all of which result from deficiencies in the hematopoietic system of the patient, may be cured by establishing a chimeric hematoimmune system in the patient using purified or enriched donor hematopoietic progenitor stem cells and cell populations which facilitate engraftment from a normal donor. The chimeric hematoimmune system should preferably be at least 10% donor origin (allogeneic or xenogeneic) since as little as 10% donor origin chimerism (allogeneic or xenogeneic) can provide adequate replacement to cure or alleviate these diseases.

The ability to establish successful xenogeneic chimerism can provide methods of treating or preventing pathogen-mediated disease states, including viral diseases in which species-specific resistance plays a role. For example, the HIV virus infects primarily the CD4⁺ T cells and antigen presenting cells produced by the bone marrow stem cells. Some animals, may possess native immunity or resistance to AIDS. By establishing a xenogeneic hematoimmune system in a human recipient, with an AIDS resistant and/or immune animal as donor, the hematopoietic and/or immunopoietic system of the human recipient can acquire the AIDS resistance and/or immunity of the donor animal. Other pathogen-mediated disease states may be cured or prevented by such a method using animals immune or resistant to the particular pathogen that causes the disease. Some examples include hepatitis A, B, C, and non-A, B, C hepatitis. Since the facilitatory cell populations play a major role in allowing engraftment of stem or progenitor cells across a species disparity, this approach will rely upon the presence of the facilitatory cell populations in the bone marrow inoculum and when necessary, the concentration of these cell populations may be adjusted such that engraftment is facilitated. Therefore, a preferred approach would be to establish the xenogeneic chimeric hematoimmune system using cellular compositions comprising purified or enriched donor cell populations which facilitate engraftment by methods disclosed herein or compositions depleted of T cells, B cells and NK cells, and reconstituted with the appropriate number of T cells (which bear the appropriate TCR on the cell surface), NK cells and/or B cells.

Furthermore, some animals, such as, for example, baboons and other non-human primates, possess native immunity or resistance to hepatitis. By transplanting a liver from a baboon or other hepatitis resistant animal into a victim of hepatitis using a method of the present invention, wherein a xenogeneic chimeric hematoimmune system is established in the victim using purified or enriched donor cell populations which facilitate engraftment plus stem cells, the donor liver will not be at risk for hepatitis, and the recipient will be tolerant of the graft, thereby eliminating the requirement for nonspecific immunosuppressive agents. Unmodified bone marrow or purified or enriched hematopoietic progenitor cells may suffice as the liver may serve as a hematopoietic tissue and may contain cell populations which facilitate engraftment that will promote the engraftment of stem cells from the same donor.

Establishing a mixed chimeric hematoimmune system has also been found to be useful in treating malignancies such as leukemia and lymphoma (Sykes et al., 1990 Proc. Natl. Acad. Sci., U.S.A., 87:5633-5637). Usually, mixed chimerism is preferred. However, fully allogeneic or fully xenogeneic chimerism may be preferred in certain instances. For example, the present invention provides a method of treating leukemia or other malignancies of the lymphohemaiopoietic system comprising substantially destroying the patient's hematoimmune system and establishing a fully allogeneic chimeric hematoimmune system by the methods described herein. Since the patient's own hematoimmune system is cancerous, it is preferred to fully replace the autologous cells with allogeneic cells of a non-cancerous donor. In this case, allogeneic purified or enriched hematopoietic progenitor cells and cell populations which facilitate engraftment may be used in order to totally eliminate all cancer cells in the donor preparation, especially if high dose chemotherapy or irradiation is used to ablate endogenous cell populations which facilitate engraftment.

The present invention also provides a method of resisting physiological effects of aging. Current research indicates aging is related to hormonal changes, such as, for example, lower growth hormone. These changes can result in decreased physiological and/or physicochemical protection, such as, for example, protection against free radicals. Using methods of the present invention, transplantation of the pituitary, pituitary and hypothalamus, or other endocrine tissues can provide renewed hormone levels.

The present invention also provides methods of practicing gene therapy. It has recently been shown that a recipient may reject sometimes even autologous cells that have been genetically modified. Utilizing methods of the present invention, a chimeric immune system can be established in a recipient using hematopoietic cells that have been genetically modified in the same way as genetic modification of other cells being transplanted therewith. This will render the recipient tolerant of the genetically modified cells, whether they are autologous, syngeneic, allogeneic or xenogeneic.

It will be appreciated that the present invention discloses cellular compositions comprising stem or progenitor cells and cell populations such as αβ TCR⁺ T cells, γδ TCR⁺ T cells and/or NK cells which facilitate engraftment. The cellular compositions can contain these cells in enriched or purified form. The compositions are first depleted of T cells, B cells, and NK cells and then reconstituted with at least one cell population that facilitates engraftment at levels determined to facilitate engraftment while minimizing the risk of GVHD. The invention also includes methods of purifying or enriching the stem cell compositions and engraftment-enhancing cell population compositions of the invention, methods of using the compositions of the invention to establish fully, completely or mixed allogeneic or xenogeneic chimeric immune systems, methods of reestablishing a syngeneic immune system, and methods of utilizing the compositions of stem cells and/or cell populations which facilitate engraftment to treat or prevent specific diseases, conditions or disorders.

### 60 EXAMPLES

Bone marrow transplantation (BMT) has become the most effective therapy for patients diagnosed with a diverse number of acute and chronic leukemias. For example, Chronic Myeloid Leukemia (CML) is a fatal disease with a median duration of 3 years for the chronic phase (Sokal, 1976, Am. J. Hematol.,1:493; Kantaijian et al., 1985, Blood, 888:1326). Despite standard therapy, most patients eventually progress to the acute phase or blast crisis with a median life expectancy of only 2 to 5 months (Coleman et al., 1980, Blood 55:29; Clarkson, 1985, J. Clin. Oncol. 3:135; Gale et al.,1992, Leukemia 6:863; Champlin et al., 1994, Bone Marrow Transplantation Ed. Forman, S. J., Blume, K. G., Thomas, E. D. (Chapter 44)). Previous studies utilizing related allogeneic BMT for treatment of CML patients demonstrated a 50 to 60% and 35 to 40% disease-free survival (DFS) rate for patients transplanted during chronic and accelerated phases, respectively (Champlin, supra). BMT is also indicated for patients diagnosed with Acute Myeloid Leukemia (AML) or Acute Lymphoblastic Leukemia (ALL) that relapsed following remission induced by conventional chemotherapy (Grever, 1987, Semin. Oncol. 14:416; Long et al., 1994, Bone Marrow Transplantation Ed. Forman, S. J., Blume, K. G., Thomas, E. D. (Chapter 45)); Doney et al., 1991, Bone Marrow Transplant 7:453). Primary chemotherapy will cure only 10-20% of adult patients with acute leukemia. While additional treatment with chemotherapy may lead to a second remission, after a relapse, such patients often relapse again within 6 months (Chao et al., 1994, Bone Marrow Transplantation Ed. Forman, S. J., Blume, K. G., Thomas, E. D. (Chapter 46)). Furthermore, patients diagnosed with ALL who are 30 years or older, have had WBC>100,000 cells/μL, cytogenetic abnormalities, or required>4 weeks to achieve first remission have a 72-82% probability of relapse within 5 years and should also be considered as candidates for BMT (Chao et al, supra). Allogeneic BMT offers these patients a chance for DFS. A recent review of transplantation with HLA-matched bone marrow donors indicated that patients diagnosed with AML in first remission or after first remission have a 50-70% or 20 to 50% chance of DFS 5 years post-BMT, respectively (Thomas, 1995, Perspectives in Biology and Medicine 38:230). Patients diagnosed with ALL in first or second remission have a 30-60% chance of DFS 5 years post-BMT, while ALL patients in relapse have only a 10-30% chance of DFS (Thomas, 1992, Seminars in Oncology 19:3). Thus, the use of BMT early in the course of disease with both chronic and acute leukemias may lead to an increased opportunity for DFS.

Unfortunately, only approximately 35% of patients can identify an HLA-identical sibling bone marrow donor (Beatty et al., 1993, Blood 81:249; Keman, 1994, Bone Marrow Transplantation Ed. Forman, S. J., Blume, K. G., Thomas, E. D. (pp. 124-135)); Armitage, 1994, New England Journal of Medicine, 330:827). Therefore, clinicians have also used grafts from partially-matched related donors and grafts from unrelated donors, which are more likely to cause GVHD. T cell depletion (TCD) is the most effective method for preventing GVHD, but has led to increased rates of graft failure/rejection and leukemic relapse. The applicants have developed technology to enable successful transplantation of partially matched related and unrelated bone marrow for patients diagnosed with both chronic and acute leukemias. The National Marrow Donor Program (NMDP) has enrolled nearly 5 million volunteers such that approximately 80% of patients can identify an HLA-A, -B, and -DR matched unrelated donor within. 80 days of initiating the search. In addition, any patient with a living biologic parent can identify a haplo-identical related donor. The present invention utilizes immunoselection techniques to eliminate cells that cause GVHD while enriching for cells that promote engraftment. This is accomplished by generating a cellular composition comprising hematopoietic stem or progenitor cells, which composition is then reconstituted with respect to specific populations of cells eliminated during immunoselection but added back at concentrations that facilitate engraftment and minimize the risk of GVHD.

### 6.1. The Clinical Trial

A clinical trial was undertaken to examine hematopoietic reconstitution in patients with hematologic malignancies treated with processed allogeneic bone marrow. The protocol accepted many poor risk patients with an expected high mortality since the important endpoints of this exploratory study were relatively short term. Specifically, the investigators were interested in the evaluation of engineered bone marrow grafts on the incidence of severe acute GVHD, where severe is defined as grades 3 and 4. Acute GVHD occurs within the first 100 days post Bone Marrow Transplant (BMT). Engraftment parameters were evaluated by determination of time to engraftment of both neutrophils and platelets. Neutrophil engraftment is usually expected within 28 days of BMT. Although four processes were utilized during the clinical investigation, the study was not designed to specifically compare process outcomes. The effect of cell dose on clinical outcome was analyzed using statistical methods known in the art, including univariate and multivariate Cox proportional hazard ratio (see Collet, D. (1994) Modelling Survival Data in Medical Research, Chapman & Hall, London; Cox, D. R. (1972), Regression Models and Life Tables (with discussion), Journal of Royal Statistical Society, B, 74:87-220), and logistic regression models (see Collet, D. (1991) Modeling Binary Data, Chapman & Hall, London) to determine the cellular composition that was associated with rapid engraftment (based on neutrophil and platelet counts) and minimal risk of GVHD.

### 6.1.1 Overall Study Design and Plan

The study was an unrandomized, uncontrolled, academic phase 1 pilot evaluation of processed allogeneic bone marrow cells used in the treatment of hematologic malignancies. More specifically, this clinical evaluation was designed to determine if leukemia patients treated with the described preparative regimen and engineered allogeneic stem cell transplant would be associated with a low rate of early treatment failure. Twenty-four patients were originally proposed for treatment under the protocol; these were subjects that had largely poor prognosis diseases with high-risk transplants. Transplants were considered high-risk because of recipient age and/or the degree of HLA mismatch between the donor and recipient.

The study design became four sequential treatment cohorts as the trial investigated the effect of graft modification on short-term patient outcomes. Treatment cohorts were defined by graft composition. Eventually, a total of 83 patients were treated.

The trial was terminated when the last living patient to be treated reached 365 days post-bone marrow transplant date. For the purpose of study closeout, individual clinical sites provided patient status information where possible for patients living beyond the one year of study follow-up.

### 6.2. Statistical Methods

The analysis group included all of the patients who were originally enrolled in the trial as described supra. All patients in the trial as described above received bone marrow processed under processes 1 through 4 described infra.

The concentration of total nucleated cells (TNC), CD34⁺ cells, αβ TCR⁺ T cells, γδ TCR⁺ T cells, B cells, total T cells, FC, and NK cells in the processed bone marrow was summarized for each process by calculating the mean, median, standard deviation, and range. Box plots graphically display the data. Cumulative probability distributions for time to event endpoints were generated by the Kaplan-Meier product-limit method (Collet, 1994, supra). Patients who failed to achieve the event of interest were right censored in the analysis (i.e., all patients enrolled in the study were included in the calculation of parameter estimates up until the time of their removal from the study). Ninety-five percent confidence intervals were calculated for the estimated median time to event. Comparisons were made using the log-rank test (Collet, 1994, supra). All tests of statistical significance were two-sided and assessed using a type I error rate of 5% (i.e., α=0.05). No adjustments were made to control the overall type I error rate associated with multiple tests of significance.

The effects of prognostic variables and covariates on the time to engraftment and the incidence of aGVHD were assessed using Cox proportional hazards and logistic regression models, respectively. The effect of each prognostic variable or covariate was considered significant if the addition of the variable or covariate in the model resulted in a statistically significant reduction in the value of the −2 log likelihood statistic at the α=0.05 level. Terms were added to the model in a forward stepwise manner. An interaction between terms was declared if the addition of the interaction term in the model resulted in a significant reduction in the−2 log likelihood statistic at the α=0.05 level.

### 6.3. The Patients

All patients enrolled in the study generally fulfilled the criteria described below:
1. Patients were aged 18 months to 60 years.
2. Patients diagnosed with acute leukemia of any type after relapse or chronic lymphocytic leukemia (CLL) after relapse; or those with acute leukemia who failed to respond to induction chemotherapy; or those with chronic myelogenous leukemia (CML) in the accelerated or blastic phase.
3. Patients diagnosed with acute myelogenous leukemia (AML) or acute lymphocytic leukemia (ALL) with high-risk chromosome abnormalities such as−5, −7, +8, t(9:22), t(8:14); or with CML in the late chronic phase (>1 year).
4. Patients diagnosed with myelodysplasia and granulocytes<0.5×10⁹/L or platelets<50×10⁹/L; or patients diagnosed with myelodysplasia and have high-risk cytogenetic abnormalities such as+8, −7 or−5.
5. Patients diagnosed with lymphoma or myeloma, after failure of initial therapy or relapse.
6. All potential patients were between the ages of 18 months and 60 years with an unrelated donor (matched or mismatched for one BLA A, B or DR locus) or a related donor (phenotypically 1, 2 or 3 antigen mismatched).
7. Patients meeting the eligibility requirements above had adequate hepatic and renal function demonstrated by pre-transplant lab values of bilirubin of≦1.5 mg/dL, SGPT and SGOT≦2.5× the upper limits of normal and serum creatinine≦1.5 mg/dL.
8. Patients had adequate cardiopulmonary function as documented by a left ventricular ejection fraction≦45% (without inotropic support or within normal limits per institutional criteria) and pulmonary function (DLGO and FEV₁)≦60% predicted for age and size.

Patients were not enrolled into this clinical study if it was determined that they met one or more of the exclusion criteria listed below:
1. Patients had received chemotherapy, immunotherapy, hormonal or radiation therapy within three weeks of entry into the study. All potential patients who had been treated by one of these therapies must have recovered from the side effect(s) prior to entry into the study.
2. Patients had received total body irradiation (TBI) as part of a previous treatment and/or therapy.
3. Patients had a life expectancy of less than 12 weeks.
4. Patients had a performance status of<70 on the Karnofsky Scale or the Lansky Scale.
5. Patients had a serious intercurrent medical illness such as uncontrolled diabetes mellitus, uncontrolled and/or active infection, uncontrolled CNS leukemia or who, in the judgment of the investigators, could not tolerate a bone marrow transplant.
6. Patients had renal insufficiency, as indicated by a serum creatinine level greater than 1.5 mg/ml.
7. Patients had hepatic insufficiency, as indicated by a serum albumin level less than 3.0 gm/dL; or acute hepatopathy as indicated by a SGPT or SGOT greater than 2.5 times the upper limit of normal -or total bilirubin>1.5 mg/dL.
8. Patients had pulmonary insufficiency, as indicated by a measure Forced Vital Capacity (FVC) or D_{L}CO of<60% of predicted.
9. Patients of child bearing potential who were not practicing adequate contraception as defined by the investigators at the respective sites.
10. Female patients of child bearing potential who were pregnant as indicated by a positive result with a serum HCG test. (Exceptions to the above criteria were permitted at the discretion of the investigator)

### 6.4. Patient Preparation for Transplant

Thiotepa (5 mg/kg) as a 2 hour intravenous (IV) infusion was administered on day−6.

Cyclophosphamide was administered at a dose of 60 mg/kg in 300 ml of 5% dextrose solution over 2 hours on each of 2 consecutive days (Days−5 and−4). Antiemetics were to be administered prior to the first dose and continued on a fixed schedule through 12-24 hours after the last dose. Intravenous fluids were administered as maximally tolerated beginning 4 hours prior to the first dose of cyclophosphamide through 24 hours after the last dose, and 10-20 mg Lasix was permitted IV 1 and 6 hours after each dose of cyclophosphamide.

Antithymocyte globulin (ATG) (Upjohn) was administered at a dose of 15 mg/kg intravenously over 4 hours on days−4 and−2. An equivalent dose was permitted if using a different brand of ATG only if the patient was hyper-sensitive to horse ATG. It was permitted to premedicate with acetaminophen, diphenhyrdamine and methylprednisolone.

Total Body Irradiation at 3 Gy per day given on days−3, −2, −1 and 0 for a total of 12 Gy. Lung shielding (5 half value layers) was employed for one fraction.

Methylprednisolone was administered as an TV infusion over 1 hour beginning on day−2 and completed on day 0. The dose was 0.5 g/kg every 12 hours for a total of four doses.

Tacrolimus was given at a dose to maintain a level of 5-15 ng/ml through Day+180 (IV while hospitalized, PO once discharged). After Day+180, the dosage was to be reduced by 20% every 2 weeks thereafter, as tolerated, and discontinued when the dose reached 1 mg/day.

The transplant was infused according to each participating institution standard (i.e., 30 minutes into the central line).

### 6.5. Marrow Donor Evaluation

All donors participating in this clinical trial were evaluated prior to their bone marrow harvest. The evaluation included, but was not limited to the following tests:
1. CBC and differential, platelets.
2. CMV antibody titer.
3. Antibodies to HIV, HTLV-I, HCV, and Hepatitis B (and HbsAg).
4. HLA A, B, C and DR, DQ, DP by serology, flow cytometry and/or molecular analysis.

### 6.6. Processing of Bone Marrow Cells for Transplant

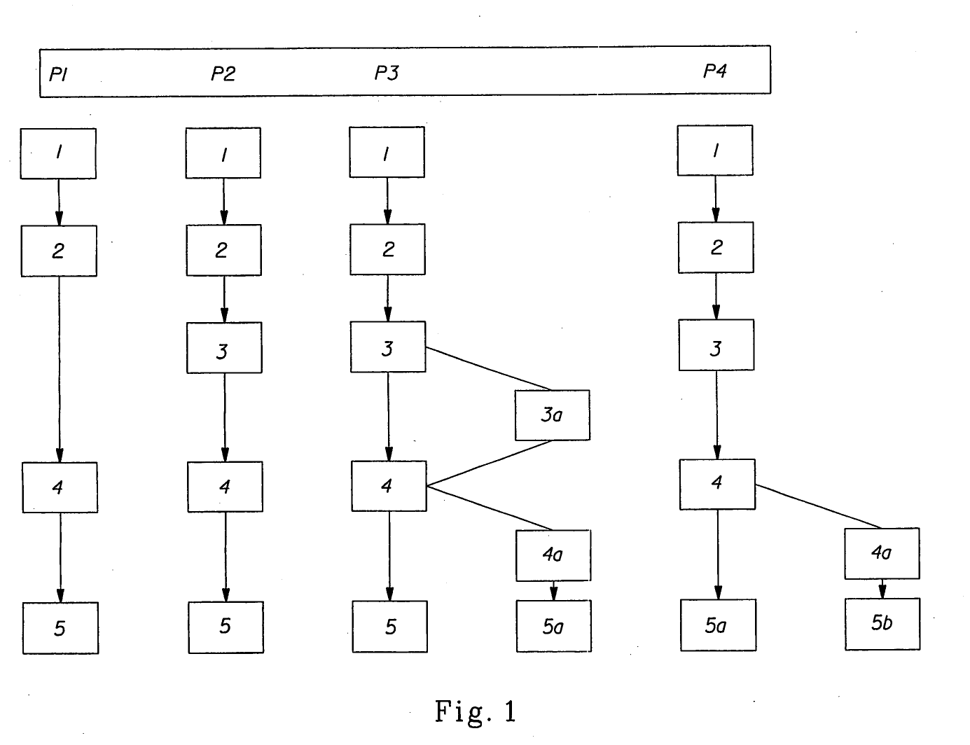

The following steps summarize the procedure used to process cells derived from bone marrow or peripheral blood used in processes 1, 2, 3, and 4 of this trial (FIG. 1). Process 1 combined donor bone marrow and mobilized peripheral blood products. All other processes used only bone marrow cells. The goal of process 1 was to concentrate facilitating cells (Kaufman et al., supra) and CD34⁺ cells while depleting T cells, NK cells and B cells. An additional bead depletion step was added in Process 2 to further deplete the αβ TCR⁺ T cells. Details regarding steps 2, 3, and 4 are provided in Sections 6.7, 6.8, and 6.9, respectively.
1. Donor bone marrow or mobilized peripheral blood was harvested and sent to Chimeric Therapies, Inc.
2. A mononuclear cell layer was prepared using the COBE 2991™ (COBE BLT, Inc. Lakewood, Colo.) and density gradient separation.
3. Depletion with anti-qffCR MAb: cells were incubated with anti-αβTCR⁺ T cells MAb antibody, washed (3×), incubated with Dynal ferromagnetic beads, and then were separated with MAXSEP® system (Baxter Healthcare Corp., Biotech Group, Immunotherapy Division, Deerfield, Ill.).
3a. First Pass Rinse: cells were rinsed in the bag after immunomagnetic depletion step to recover cells within the system.
4. Immunomagnetic depletion with MAb cocktail: cells were incubated with anti-αβTCR, —CD56, and —CD 19 MAbs, washed (3×), incubated with Dynal ferromagnetic beads, and then were separated with MAXSEP®. Cells recovered from this step were collected in the Primary bag.
4a. Second Pass Rinse: cells were rinsed in the bag after magnetic depletion step to recover cells within the system.
5. Primary Bag: Cells recovered from the second immunomagnetic depletion step utilizing the antibody cocktail were transferred into the shipping media and collected in the Primary Bag. This was shipped to the patient.
5a. Satellite Bag: Cells from the Second Pass Rinse were transferred into shipping media and collected in the Satellite Bag. This was shipped to the patient. It was used to increase the CD34⁺ cell content of the final cellular composition product.
5b. Enriched αβCR⁺ T Cell Component: Cells from the First Pass Rinse were transferred into shipping media and collected in the Enriched αβCR⁺ T cells component bag. These cells were shipped to the patient and were used to adjust the αβTCR⁺ T cell content of the final product.
(Note: the depletion cocktail used in Process 2, and for some patients in Process 3 and 4 included anti-TCRγδ MAb.)

### 6.7. Step Gradient Centrifugation to Remove RBC, Platelets and Cell Debris

Bone marrow obtained from donors was originally processed using the COBE 2991™ to reduce sample volume and remove red blood cells. Mononucleated cells were then obtained by density gradient according to the following protocol. Individual conical centrifuge tubes containing step gradients were prepared by adding 15 mL of CELL/FLEX-1053C (Atlantic Biologicals, Norcross, Ga.) into each tube. A volume of 15 mL of CELL/FLEX-1077C (Atlantic Biologicals, Norcross, Ga.) was underlayed in the bottom layer of each step gradient tube. A volume of 15 mL of bone marrow cell suspension was layered on the top layer of each gradient tube. The tubes were centrifuged at 1400×g for 25 minutes at room temperature (˜22° C.). The cell layer at the middle interface of the step gradient was collected from each tube and pooled into multiple conical centrifuge tubes. Cell suspensions were diluted by adding DPBS+10 mg/mL human serum albumin (HSA) to each tube. The cell suspensions were centrifuged at 900×g for 10 minutes at 4° C. The supernatants were discarded and the cell pellets were re-suspended with DPBS+10 mg/mL HSA. The cell suspensions were centrifuged at 900×g for 10 minutes at 4° C. The supernatants were discarded and the re-suspension and centrifugation steps were repeated one more time to remove residual density gradient material. The washed cell pellets were re-suspended in DPBS+10 mg/mL HSA to a total volume of 125 mL. Samples were obtained for quality control analysis. Quality control testing included (a) TNC, (b) viability analysis via trypan blue dye exclusion and (c) flow cytometry.

### 6.8. Immunomagnetic Separation of αβ TCR (+) T CELLS, Generation of Enriched αβ TCR⁺ T Cell Component Cell Product

The TNC content of the bone marrow cell suspension was typically below 1×10¹⁰ after the step gradient centrifugation step. TCR+ αβ BMA 031 MAb (Aventis Pharma, Marburg, Germany) was added to the cell suspension at a ratio of 2.5 mg per 1×10¹⁰ cells. This material was provided as a sterile solution and was next added aseptically to the cell suspension. The volume of the cell suspension was adjusted to 200 mL through the addition of DPBS+10 mg/mL HSA. The cell suspension was transferred to a sterile transfer bag and incubated for 45 minutes at 4° C. on a laboratory mixer. The MAb was allowed to bind to the target T lymphocytes expressing TCR αβ during this period. The cell suspension was transferred to sterile conical centrifuge tubes and diluted 1:1 with DPBS+10 mg/mL HSA. The cell suspension was centrifuged at 330×g at 4° C. for 10 minutes. The supernatant was discarded and the cell pellet was re-suspended in DPBS+10 mg/mL HSA. The centrifugation and re-suspension steps were repeated two more times to remove unbound MAb. The washed cell pellet was re-suspended in DPBS+10 mg/mL HSA to a volume less than or equal to the bead incubation reference volume (see below).

A sufficient quantity of DYNABEADS® 450 sheep anti-Mouse IgG (DYNABEADS) (Dynal Biotech, Oslo, Norway) for both immunomagnetic depletion steps of the procedure were washed with DPBS followed by DPBS+10 mg/mL HSA. The cell suspension was transferred to a sterile transfer bag. DYNABEADS® were added to the cell suspension at a bead to cell ratio in the range of about 2:1 to about 12:1 in the αβ TCR⁺ T cell depletion step. The remainder of the beads was stored at 4° C. for use in the second immunomagnetic depletion step. The bead to cell ratio in this step was approximately 1:1. The volume of bead/cell mixture was adjusted to ensure a concentration of at least 2×10⁷ beads/mL by the addition of DPBS+10 mg/mL HSA. The cell suspension/DYNBEAD® mixture was incubated on a laboratory mixer for 45 minutes at 4° C. Target αβTCR⁺ T lymphocytes sensitized with MAb were allowed to bind to the DYNABEADS during this period.

The MAXSEP® Magnetic Cell Separation System was primed with Sodium Chloride 0.9% Injection+10 mg/mL HSA. The transfer bag containing the cells and DYNABEADS was then connected to the MAXSEP® system. The pump was activated, causing the cell/bead mixture to travel through the primary and secondary magnetic chambers, where the DYNABEADS® along with their attached target cell population (αβ TCR⁺ T cells) were separated from the free unbound cells. The effluent containing these cells was collected into a recovery bag. The bag was disconnected from the system. A sample was obtained from this bag for TNC and flow cytometric analysis.

The primary container of the MAXSEP® disposable set was removed from the system. Approximately 125-150 ml of Sodium Chloride 0.9% injection+10 mg/mL HSA was fed by gravity into the primary container. The primary container was mixed for 5 minutes on a laboratory mixer. The primary container was placed back on the MAXSEP® system. The pump was activated, causing additional unbound cells to travel through the primary and secondary magnetic chambers and into a separate collection bag. This “rinse” bag (first pass rinse) was removed from the system and a sample was obtained from this bag for a cell count and flow cytometric analysis. The cell suspension was transferred to a conical centrifuge tube and the volume was adjusted to 200 ml through the addition of DPBS+10 mg/ml HSA. The cell suspension was centrifuged at 330×g at 4° C. for 10 minutes. The supernatant was discarded and the cell pellet was resuspended to a volume of 104 ml with a defined, serum-free transport medium composed of X VIVO 10 medium (Biowhittaker, Inc., Walkersville. Md.) supplemented with 10 units/mL heparin sulfate, 25 mg/mL HSA and 10 μg/mL gentamicin sulfate. The cell product was transferred into a transfer bag. A sample equal to 4 ml was obtained for the following Quality Control tests: (a) cell counts via Coulter Counter, (b) viability analysis via trypan blue dye exclusion, (c) flow cytometry, (d) microbial bioburden analysis, (e) gram stain and (f) endotoxin quantitation. The product in this bag is referred to as the Enriched αβ TCR⁺ T cell Component.

### 6.9. Immunomagnetic Separation of αβ TCR⁺ T Cell, CD 19 (+) and CD 56 (+) Cells; Generation of Satellite Bag Cell Product

Next, a cocktail of three MAbs was added to the cells recovered in the effluent from the first immunomagnetic depletion step. Anti-CD 19 (Becton Dickenson, San Jose, Calif.) was added a concentration of 1.0 mg per 1×10¹⁰ TNC. Anti-αβ TCR (BMA031) and anti-CD 56 (Becton Dickenson, San Jose, Calif.) were added at a concentration of 2.5 mg per 1×10¹⁰ TNC. The TNC value was used to determine the monoclonal antibody quantities to add. These materials were provided as sterile solutions and were added aseptically to the bag. The cell suspension was incubated for 45 minutes at 4° C. on a laboratory mixer. The MAbs were allowed to bind to the target cell populations during this period. The cell suspension was transferred to sterile conical centrifuge tubes and diluted 1:1 with DPBS+10 mg/mL HSA. The cell suspension was centrifuged at 330×g at 4° C. for 10 minutes. The supernatant was discarded and the cell pellet was re-suspended in DPBS+10 mg/mL HSA. The centrifugation and re-suspension steps were repeated two more times to remove unbound MAb. The washed cell pellet was re-suspended in DPBS+10 mg/ML HSA to a volume less than or equal to the bead incubation reference volume (see below). The cell suspension was transferred to a sterile transfer bag.

The remaining volume of washed DYNABEADS® was added at a bead to cell ratio of 1:1 (this ratio is based on the TNC count obtained following step gradient centrifugation). The cell suspension was adjusted through the addition of DPBS and 10 mg/mL HSA to a minimum level of 2×10⁷ beads/mL. The cell suspension/DYNABEAD® mixture was incubated on a laboratory mixer for 45 minutes at 4° C. Target αβ TCR⁺ T lymphocytes, B lymphocytes and NK cells sensitized with specific MAbs were allowed to bind to DYNABEADS® during this incubation period.

The MAXSEP® system described supra was primed with Sodium Chloride 0.9% w/v Injection+10 mg/mL HSA. The transfer bag containing cells and DYNABEADS was connected to the MAXSEP® system. The pump was activated, causing the cell/bead mixture to travel through the primary and secondary magnetic chambers, where the DYNABEADS along with their attached target cell population (αβ TCR⁺ T cells, CD 19 (+) and CD 56 (+) cells) were separated from the free unbound cells. The effluent containing these cells was collected in a recovery bag that was disconnected from the system for further manipulations.

The primary container of the MAXSEP® disposable set was removed from the system. Approximately 125-150 ml of Sodium Chloride 0.9% injection+10 mg/mL HSA was fed by gravity into the primary container. The primary container was mixed for 5 minutes on a laboratory mixer. The primary container was placed back on the MAXSEP® system. The pump was activated, causing additional unbound cells to travel through the primary and secondary magnetic chambers and into a separate collection bag. This “rinse” bag was removed from the system and a sample was obtained from this bag for a cell count and flow cytometric analysis. These unbound cells are referred to as the Second Pass Rinse and the rinse bag is referred to as the Satellite Bag.

### 6.10. Preparation, Packaging and Shipment of the Final Product

The effluent cell suspension from the second immunomagnetic depletion step was transferred into 2 sterile conical centrifuge tubes and centrifuged at 330×g at 4° C. for 10 minutes. The supernatant was discarded and the cell pellets were re-suspended in a defined, serum-free transport medium composed of X VIVO 10 medium (Biowhittaker, Walkersville, Md.) supplemented with 10 units/mL heparin sulfate, 25 mg/mL HSA and 10 μg/mL gentamicin sulfate. The total volume was adjusted to 200 mL. The cell product was transferred into a cryocyte bag. A sample was obtained for the following Quality Control tests: (a) cell counts via Coulter Counter, (b) viability analysis via trypan blue dye exclusion, (c) flow cytometry, (d) microbial bioburden analysis, (e) CFU assay, (f) gram stain and (g) endotoxin quantitation. Equal volume of the cell suspension were then transferred into two transfer bags which were labeled appropriately with the necessary patient and process information. The material in these two bags was referred to as the Primary Bag product.

### 6.11. Treatments Administered

The treatments administered were either processed bone marrow alone or a combination of processed bone marrow and processed mobilized peripheral blood stem cells. Up to and including patient 15, most patients with a related donor received both processed bone marrow and processed mobilized peripheral blood stem cells and a few patients received only process bone marrow. All patients beyond patient 15 received processed bone marrow only, regardless of donor status.

Four processes for the preparation of engineered graft are discussed herein. All products were processed to deplete T, B and NK cells and retain graft-facilitating cells and stem cells. The goal of Process 2 was to further reduce the concentration of αβ TCR⁺ T cells in the graft composition compared to Process 1. A second immunomagnetic depletion step was performed using a MAb directed against the αβ TCR. Both Processes 3 and 4 employed a graft definition developed from the experience gained with the earlier processes and modifications were made to adjust the T cell dose. This was based on the observation that a minimum number of T cells were required to facilitate engraftment. In addition, an upper limit of αβ TCR⁺ T cells was identified which, when exceeded, increased the risk of severe GVHD. During Processes 3 and 4, some bone marrows were prepared using both αβTCR⁺ T cells and γδTCR⁺ T cells monoclonal antibodies and some processes used αβTCR⁺ T cells monoclonal antibody alone in the depletion.

During the use of processes 1 and 2, the goal was to infuse a minimum of 1×10⁶ CD34(+) cells/kg. During processes 3 and 4, the target graft definition was as follows:
CD34⁺ cells≧1.00×10⁶/kg
Total T cells≧1.00×10⁵/kg
Total αβ TCR⁺ T cells≦1.00×10⁵/kg

### 6.12. Results: Summary of Engraftment Data

### 6.12.1. Neutrophil Engraftment

The time to neutrophil engraftment to≧0.5×10⁹ cells/L after bone marrow transplant is summarized by process in Table 1. All patients had an absolute neutrophil count (ANC) nadir≦0.5×10⁹/L and were therefore evaluable for this analysis. Seventy-five patients (90%) achieved neutrophil engraftment. Neutrophil engraftment was not observed in eight patients; all were censored on their last day on study.

Overall, the time to neutrophil engraftment to≧0.5×10⁹/L was similar across the 4 processes (p=0.6, log-rank test). The median times to engraftment were 13 days for patients treated under process 1, 3, and 4 and 15 days for patients treated under process 2.

TABLE 1
Summary of Neutrophil Engraftment

| | Process | | | |
|---|---|---|---|---|
| | 1 | 2 | 3 | 4 |
| | (n = 17) | (n = 29) | (n = 20) | (n = 17) |
| No. (%) ANC Engrafted | 17 (100%) | 26 (90%) | 18 (90%) | 14 (82%) |
| No. (%) Censored | 0 | 3 (10%) | 2 (10%) | 3 (18%) |
| Median Days to ANC | 13 | 15 | 13 | 13 |
| Engraftment | | | | |
| 95% Confidence Interval | (10, 17) | (13, 16) | (12, 18) | (11, 15) |
| Range (days) | 9-24 | 9 to >35 | 10-29 | 9-29 |

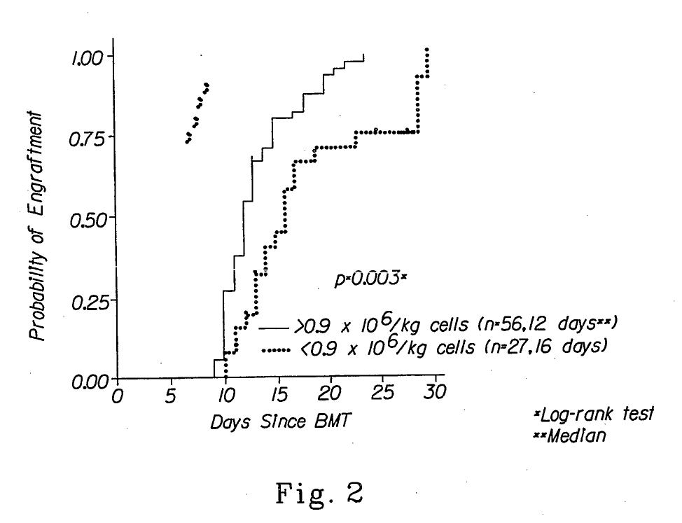

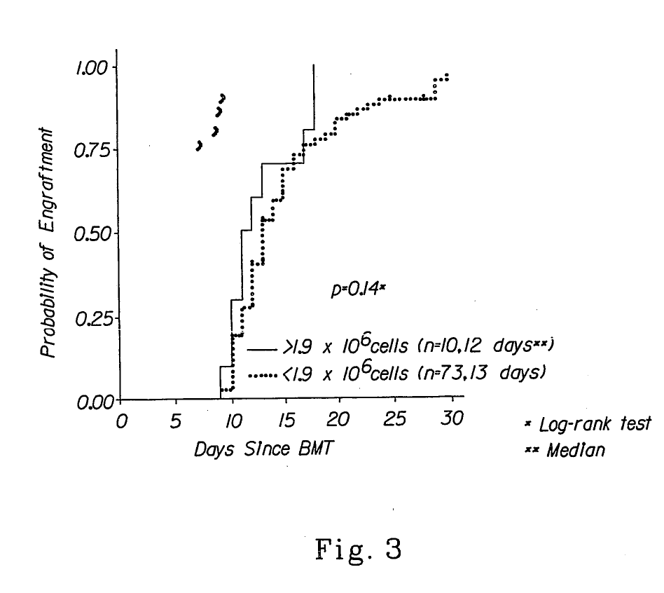

Kaplan-Meier estimates, displayed in FIGS. 2 and 3, demonstrate the relationship between CD34⁺ cell dose in the graft and the time to neutrophil engraftment.

As depicted in FIG. 2, a CD34⁺ dose of≧0.9×10⁶ cells/kg correlates positively with neutrophil engraftment. Neutrophil engraftment was defined as the first of 3 consecutive days, following bone marrow transplant, during which the neutrophil counts were greater than or equal to 0.5×10⁹/L. All patients in the data set were divided into two groups based upon the CD34⁺ cell content of their graft. The group of patients (27) that had less than 0.9×10⁶ CD34⁺ cells/kg had a median time to neutrophil engraftment of 16 days. This compared to 12 days to neutrophil engraftment for the group of patients (56) that had more than 0.9×10⁶ CD34⁺ cells/kg. The difference in the times to neutrophil engraftment was statistically significant (p=0.003). Division into these two groups did not take into account the T cell content of the graft.

Statistical analysis of CD34⁺ cell dose (FIG. 3) indicates a greater probability of engraftment at later time points with a cell dose>1.9×10⁶ cells/kg. However, when the patient data set was divided into two groups based upon a CD34⁺ cell dose above (10 patients) or below (73 patients a 1.9×10⁶ cells/kg cut off point, the difference in times to neutrophil engraftment (12 versus 13 days) was not statistically significant (p=0.14). Although the times to engraftment were not statistically significantly different, patients in the lower CD34⁺ cell group had a 25% probability of achieving neutrophil engraftment in a later timeframe (15 to 30 days post bone marrow transplant) compared with the other group. Patients in the higher CD34⁺ cell group all achieved neutrophil engraftment by 18 days post bone marrow transplant. Division into these two groups did not take into account the T cell content of the graft.

These results indicate that a CD34⁺ cell count of≧0.9×10⁶ cells/kg IBW provides a positive effect in producing shorter times to neutrophil engraftment.

### 6.12.2. Platelet Engraftment

The time to platelet engraftment to≧20×10⁹/L after bone marrow transplant is summarized by process in Table 2. All patients had a platelet count<20×10⁹/L and were therefore evaluable for this analysis. Thirty-nine patients (47%) achieved platelet engraftment. Platelet engraftment was not observed in forty-four patients; all were censored on their last day on study.

Overall, the time to platelet engraftment to ≧20×10⁹/L differed among the 4 processes (p=0.08, log-rank test). For patients treated under process 3 and 4, the median time to platelet engraftment was 28 and 31 days, respectively. The median time to platelet engraftment was determined by Kaplan-Meier distribution. When used to evaluate process 1 the median time to platelet engraftment was 196 days but this was caused by one outlying patient and thus may not accurately represent the true median for that process. The median time to platelet engraftment for process 2 was 63 days.

TABLE 2
Summary of Platelet Engraftment

| | Process | | | |
|---|---|---|---|---|
| | 1 | 2 | 3 | 4 |
| | (n = 17) | (n = 29) | (n = 20) | (n = 17) |
| No. (%) PLT Engrafted | 6 (35%) | 12 (41%) | 11 (55%) | 10 (59%) |
| No. (%) Censored | 11 (65%) | 17 (59%) | 9 (45%) | 7 (41%) |
| Median Days to PLT | 196 | 63 | 31 | 28 |
| Engraftment | | | | |
| 95% Confidence Interval | (13, 196) | (37, NC) | (18, NC) | (16, NC) |
| Range (days) | 10 to 196 | 14 to >175 | 13 to >109 | 13 to >176 |

*NC: not calculated

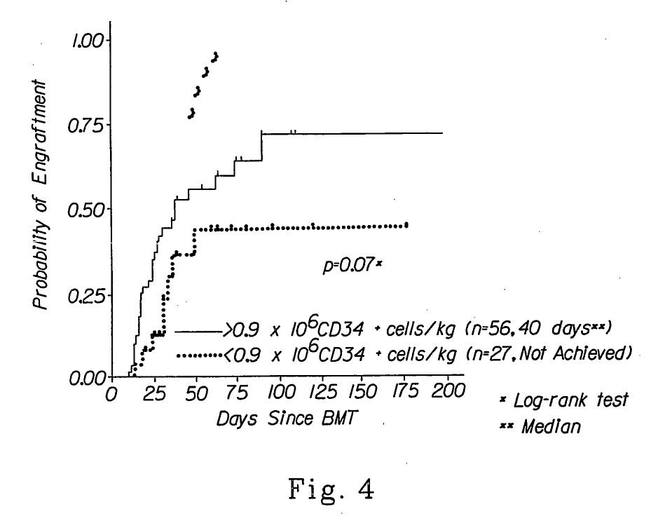

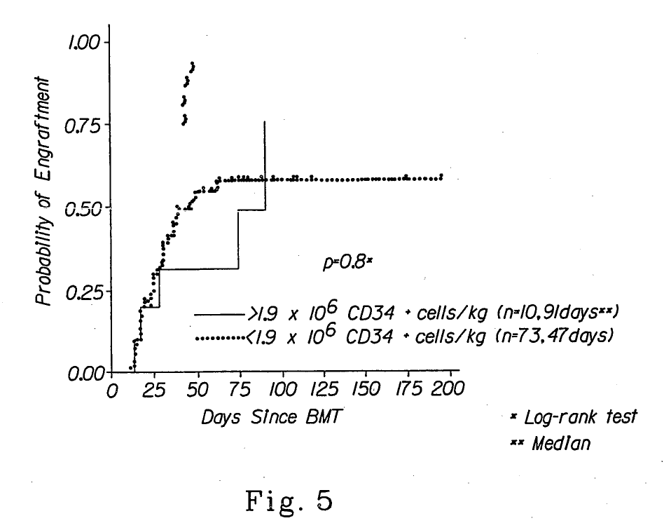

Kaplan-Meier estimates are displayed in FIGS. 4 and 5 and demonstrate the relationship between CD34⁺ cell dose in the graft and the time to platelet engraftment. The results indicate that a CD34⁺ cell dose of≧0.9×10⁶ demonstrates a positive association with respect to shorter time to platelet engraftment.

### 6.12.3. αβ T Cell Dose Affects Neutrophil Engraftment

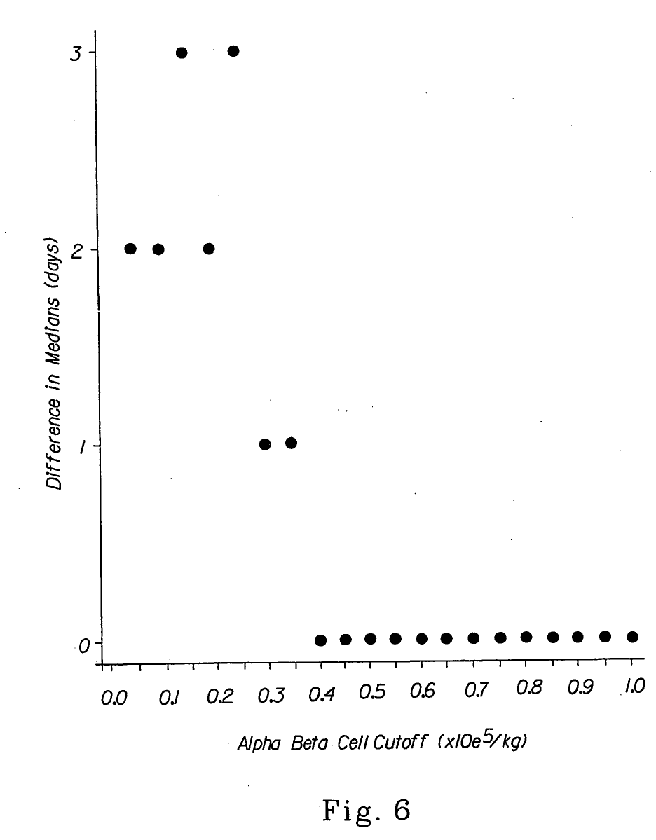

As shown in FIG. 6, increased αβ TCR⁺ T cell dose correlates with increased neutrophil engraftment (FIG. 6). Analysis of the same data set that was analyzed above for the effect of CD34⁺ cell dose on neutrophil engraftment was analyzed for the effect of αβ TCR⁺ T cell dose on neutrophil engraftment. The data shows that these cells impact time to neutrophil engraftment. The data indicates that the positive effects of αβ TCR⁺ T cells on neutrophil engraftment begin to plateau at a cell dose of 0.4×10⁵ cells/kg. They-axis of FIG. 6 shows the difference between two groups, expressed in days, when comparing the median time taken to achieve neutrophil engraftment. The groups involved in th comparison were derived from the cell content of the graft. The whole data set was divided according to a specific cut off point, which is shown on the x-axis. When the data were separated into two groups based upon an αβ TCR⁺ T cell dose above or below 0.1 5×10⁵ αβ TCR⁺ T cells/kg, the difference in median times to engraftment between the two groups was 3 days. The difference falls to zero days when two groups are compared that either have more than or less than 0.4×10⁵ αβ TCR⁺ T cells/kg. The implication is that further addition of αβ TCR⁺ T cells has little effect on the time to engraftment outcome. The Cox proportional hazards models for engraftment substantiate that both αβ TCR⁺ T cells and CD34⁺ cells have a statistically significant effect upon reducing the time taken to achieve neutrophil engraftment.

### 6.12.4. Cox Proportional Hazards Models for Engraftment

The effect of the graft composition, as measured by the cellular content in the processed bone marrow, on neutrophil and platelet engraftment was assessed using univariate and multivariate Cox proportional hazards models. Terms representing the following cells, measured in units of 10⁶/kg, were evaluated in each model: TNC, CD34⁺, αβ, γδ, B, and NK.

The results from the univariate models for neutrophil engraftment are shown in Table 3, below. Higher concentrations of TNC, CD34⁺ cells, αβ TCR⁺ T cells, and NK cells were significantly associated with earlier neutrophil engraftment (p<0.05, chi square test). Specifically, for every one unit increase in each of these cells, there was a corresponding increase in the probability of neutrophil engraftment equal to the hazard ratio in Table 3.

TABLE 3
Univariate Analysis of Neutrophil Engraftment

| Cell (×10⁶/kg | Relative Risk | 95% CI | p-value |
|---|---|---|---|
| TNC | 1.01 | (1.005, 1.02) | 0.003 |
| CD34+ | 2.07 | (1.36, 3.14) | 0.0006 |
| αβ | 2.43 | (1.29, 4.58) | 0.006 |
| γδ | 1.15 | (0.73, 1.81) | 0.5 |
| B | 1.89 | (0.76, 4.72) | 0.2 |
| NK | 1.46 | (1.05, 2.02) | 0.02 |

A multivariate Cox proportional hazards model also was used to assess the effect of graft composition on neutrophil engraftment. The model included a term for each cellular component listed above, irrespective of its relative contribution to the model, as determined by the corresponding reduction in log likelihood function. The results from this analysis are displayed in Table 4.

Similar to the univariate results, higher concentrations of CD34+ cells and αβ TCR⁺ T cells in the processed graft were significantly associated with earlier neutrophil engraftment (p<0.05 chi square test). However, unlike in the univariate analysis, TNC and NK cells were not statistically significantly associated with neutrophil engraftment in the multivariate analysis. The difference regarding the statistical significance of NK cells in the univariate model versus the multivariate model occurs because at the concentration currently used in the final graft the effect of the NK cell is minor when compared with that of the αβ TCR⁺ T cell effect.

TABLE 4
Multivariate Analysis of Neutrophil Engraftment

| Cell (×10⁶/kg | Relative Risk | 95% CI | p-value |
|---|---|---|---|
| TNC | 1.003 | (0.98, 1.02) | 0.7 |
| CD34+ | 3.18 | (1.57, 6.19) | 0.001 |
| αβ | 3.95 | (1.07, 14.53) | 0.04 |
| γδ | 2.69 | (0.44, 16.42) | 0.3 |
| B | 0.14 | (0.001, 17.05) | 0.4 |
| NK | 0.97 | (0.52, 1.79) | 0.9 |

Similar analyses were done to assess the effect of the graft composition on platelet engraftment. In the univariate analysis, none of variables tested were statistically significantly associated with platelet engraftment (Table 5).

TABLE 5
Univariate Analysis of Platelet Engraftment

| Cell (×10⁶/kg | Relative Risk | 95% CI | p-value |
|---|---|---|---|
| TNC | 1.001 | (0.99, 1.01) | 0.9 |
| CD34+ | 1.29 | (0.77, 2.18) | 0.3 |
| αβ | 1.10 | (0.51, 2.36) | 0.8 |
| γδ | 1.18 | (0.55, 2.48) | 0.6 |
| B | 0.84 | (0.14, 5.16) | 0.8 |
| NK | 1.01 | (0.68, 1.52) | 0.9 |

However, in the multivariate analysis, higher concentrations of CD34⁺ cells and αβ TCR⁺ T cells were significantly associated with earlier platelet engraftment (Table 6). For every one unit increase in each of these cells, there was a corresponding increase in the probability of platelet engraftment equal to the hazard ratio in Table 6.

TABLE 6
Multivariate Analysis of Platelet Engraftment

| Cell (×10⁶/kg | Relative Risk | 95% CI | p-value |
|---|---|---|---|
| TNC | 1.003 | (0.98, 1.03) | 0.8 |
| CD34+ | 2.20 | (1.02, 4.75) | 0.04 |
| αβ | 14.17 | (1.36, 147.80) | 0.03 |
| γδ | 3.60 | (0.88, 14.65) | 0.07 |
| B | 0.006 | (0.00, 8.91) | 0.1 |
| NK | 0.71 | (0.27, 1.83) | 0.5 |

### 6.12.5. The Incidence of Severe Acute GVHD Can be Minimized by Limiting the αβ T Cell Dose in the Graft

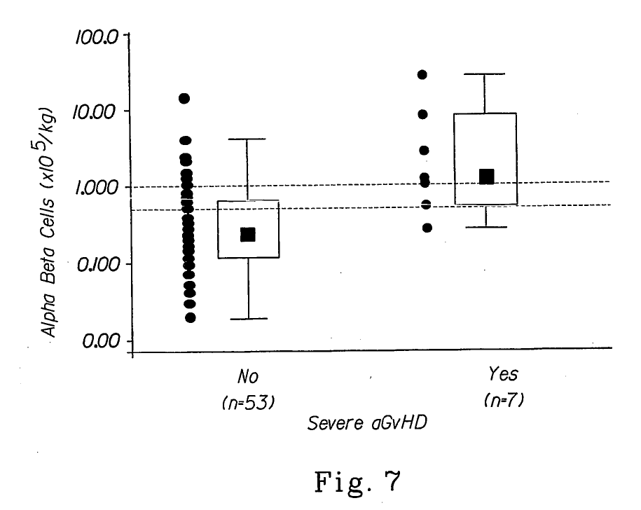

As depicted in FIG. 7, the risk of incurring severe acute GVHD increases as the αβ TCR⁺ T cell dose in the graft increases. Seven patients in the data set experienced severe acute GVHD and the median αβ TCR⁺ T cell dose received by this group was 1.14×10⁵ cells/kg IBW. Severe acute GVHD was defined as either grade 3 or grade 4. In the remainder of the patients (n=53) the median αβ TCR⁺ T cell dose received was 0.23×10⁵ cells/kg IBW and these patients did not experience severe acute GVHD. The two broken horizontal lines across FIG. 7 at 0.5×10⁵ and 1.0×10⁵ As cells/kg IBW represent the preferred cell dose range of αβ TCR⁺ T cells to be used in the methods of the invention. This range represents conditions where the incidence of serious acute GVHD should be reduced by 50% or more in a group of patients that received an engineered stem cell preparation of the invention compared with unmodified source material. A reduction of 50% or more of severe acute GVHD is considered to be a beneficial outcome. In this data set, all 60 patients received an engineered stem cell preparation of the invention, but the group of patients (n=53) that did not experience any serious GVHD tended to have more αβ TCR⁺ T cells removed.

### 6.13 Discussion

Statistical analysis performed on all patients treated in the clinical trial by processes 1-4, combined, demonstrated that there are several cell populations that are important in promoting engraftment. While it had been previously believed that T cell depletion would remove the propensity for the patient to develop acute GVHD, it also caused a high rate of graft failure. Thus T cells are recognized to be a required component within an engineered stem cell graft. According to the present invention, certain specific T cell sub-types, in defined numbers, have a therapeutic window when producing their beneficial effect. It was discovered that facilitating cells and αβ TCR⁺ T cells are beneficial in facilitating engraftment at concentrations between about 0.1×10⁵ cells/kg IBW and 1.0×10⁵ cells/kg IBW. At this concentration, a reduction in the incidence of severe acute GVHD is observed when compared to unprocessed stem cell preparations. Furthermore, it is believed that greater numbers of γδ TCR⁺ T cells than the therapeutic range of facilitating cells and αβ TCR⁺ T cells will also facilitate engraftment without adverse acute GVHD. Additionally, CD34⁺ cells at a dose of about≧0.9×10⁶ cells/kg IBW were found to be sufficient to cause engraftment when combined with appropriate doses of other cells (e.g., αβ TCR⁺ T cells and γδ TCR⁺ T cells) in the graft. CD34⁺ cell doses around 1.9×10⁶ cells/kg IBW supplemented with appropriate amounts of various cellular subsets optimize engraftment. The use of defined cellular subset dosing obviates the need for megadoses of highly purified facilitating cell and CD34⁺ cell preparations.

### 7. A PREFERRED METHOD FOR OBTAINING CELL COMPOSITIONS OF THE INVENTION

First, in order to accurately dose αβ TCR⁺ T cells within the range specified by the specified graft definition, a rinse of the first immunomagnetic depletion (prior to which the cells had been exposed to the anti αβ TCR antibody only) was employed. This rinse recovered αβ TCR⁺ T cells that were not tightly bound to the DYNABEADS®. Thus, αβ TCR⁺ T cells previously exposed to antibody could be added back to the primary product to obtain the stipulated dose of 0.5-1.0×10⁵ αβ TCR⁺ T cells/kg IBW in the final graft product.

Secondly, in order to maximize recovery of cell populations which facilitate achieving desirable clinical endpoints including engraftment and to minimize the presence of contaminating cell populations and other impurities resulting from bone marrow processing, a new protocol for enriching mononuclear cells derived from bone marrow was devised.

### 7.1. Introduction

The purpose of bone marrow transplantation is to provide the recipient with a new, permanently functioning hematopoietic system. Successful bone marrow transplantation requires that an adequate number of pluripotent stem cells be collected and infused to ensure engraftment after marrow ablative chemoradiotherapy. Current harvest techniques require multiple bone marrow aspirates to obtain a sufficient number of stem cells. A large number of peripheral blood cells are collected in addition to the pluripotent and committed progenitor cells in the harvest procedure. Contamination of the bone marrow cell population with erythrocyte, platelets, granulocytes and other cellular debris necessitates that the mononuclear cell population be isolated from this mixture prior to the immunomagnetic depletion steps described supra in Section 6.

Thus, the cell processing procedure, described supra, employed two steps upstream of the immunomagnetic depletion steps. The first step employed a COBE 2991™ (COBE BLT, Inc., Lakewood, Colo.) blood cell processor to reduce the overall bone marrow volume through the removal of plasma and a large portion of erythrocytes. The second step employed gradient centrifugation to remove the remaining erythrocytes, platelets, granulocytes, and cellular debris to produce a mononuclear cell population. The separation media used in the gradient centrifugation process were CELL/FLEX-1053C and CELL/FLEX-1077C, produced by Atlantic Biologicals, Inc. (Norcross, Ga.).

According to a preferred embodiment, a blood component separator, the COBE Spectra™ Apheresis system (COBE BLT, Inc. Lakewood, Colo.) was used to isolate mononuclear cells from human bone marrow. The employment of the COBE Spectra™ Apheresis system avoided the use of a density gradient material such as CELL/FLEX and eliminated the need for volume reduction with the COBE 2991™. The COBE Spectra™ system uses a centrifugal method to separate whole blood or bone marrow into its major components: erythrocytes, leukocytes, thrombocytes, and plasma. Anticoagulated bone marrow is pumped into the system channel while the channel is rotating clockwise in the centrifuge. This causes the component of the highest density (erythrocytes) to go to the outer wall of the channel, with layers of components proceeding toward the inner wall where the lowest density component (plasma) gravitates. Several outlet tubes are located in the channel. Flow from these tubes allows for the collection of specific components, including mononuclear cells, from the bone marrow.

Employing the COBE Spectra™ system in the bone marrow processing procedure has several advantages. The system replaces two processing steps with one and therefore shortens processing time while decreasing the quality control testing required. The procedure utilizes sterile, non-pyrogenic, single-use disposable sets in a closed system, thereby affording a higher level of process asepsis. The system also alleviates the need for a density gradient separation medium.

In addition to the implementation of the COBE Spectra™ system, the COBE 2991™ blood cell processing system was also employed to wash cells after the monoclonal antibody incubation and immunomagnetic depletion steps. The COBE 2991™ system is designed to wash cells (i.e. red blood cells units) intended for transfusion. Incorporating the COBE 2991™ system to wash cells during these process steps improved process efficiency and process asepsis since this system also employs sterile, non-pyrogenic, single-use disposable sets in a closed system.

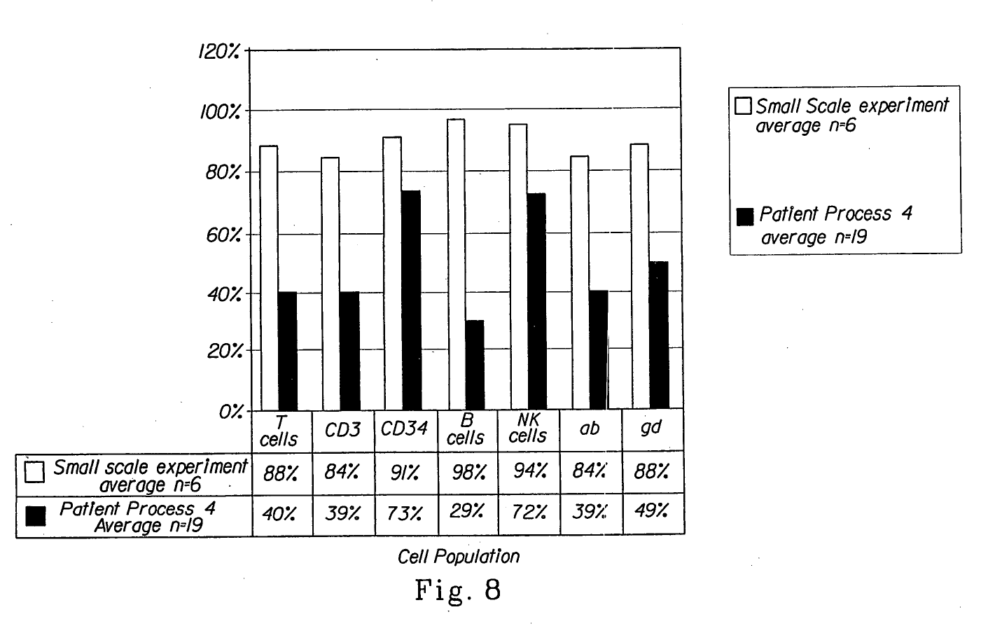

Experiments using the COBE Spectra™ to process six small scale bone marrow preparations and five mobilized peripheral blood stem cell (MPBSC) collections were performed and the results were compared with the average recovery of the large scale bone marrow processes for Process 4 patients (n=19) from the gradient centrifugation step described supra. See, e.g., FIG. 8. The results are discussed in Section 7.3, infra.

### 7.2. Materials and Methods

### 7.2.1. Harvest

Small scale bone marrow harvests (100 ml Iliac Crest bone marrow) were obtained from normal healthy donors (Poietic Technologies, Gaithersburg, Md.; AllCells, L.L.C. Berkley, Calif.). Bone Marrow (BM) from Poeitic Technologies was shipped in Hanks Buffered Salt Solution containing 0.5% BSA and 5 mM EDTA. Bone Marrow from AllCells was shipped in Phosphate Buffered Solution containing 25 U/ml Heparin.

Type O packed red blood cells were obtained from healthy donors (Biological Specialty Corporation, Colmar, Pa.).

Mobilized Peripheral Blood Stem Cell collections (MPBSC) were obtained from healthy donors (AllCells, L.L.C. Berkley, Calif.). MPBSC are shipped in Autologous Plasma and 10% Anticoagulant Citrate Dextrose Solution USP Formula A (ACDA).

100 ml of marrow was obtained and washed on the COBE 2991™ blood cell washer in order to remove any plasma. This was then combined with approximately 150 ml of red blood cells from a unit of type O packed red blood cells which had been washed on the COBE 2991 blood cells washer to remove plasma and residual white blood cells.

It was necessary to add the red blood cells because the 100 ml of marrow did not contain sufficient volume of red blood cells to run on the COBE Spectra™.

The pooled BM and RBCs were then diluted with media (DPBS with 0.96% HSA) to bring the volume up to 600-800 ml.

The same procedure was performed with the MPBSC.

### 7.2.2. COBE Spectra™ Separation

Table 7 summarizes the materials used in the COBE Spectra™ system.

TABLE 7
Materials Used in COBE Spectra ™

| Material | Manufacturer |
|---|---|
| COBE Spectra ™ | Gambro BCT, (Lakewood, CO). |
| Heat Sealer | Sebra, (Tucson, AZ) |
| ACDA | Baxter Fenwall, IL |
| Bone Marrow Processing Set | Gambro, BCT, (Lakewood, CO.) |
| Disposable WBC Tubing Set | Gambro, BCT, (Lakewood, CO.) |
| 1000 ml Saline | Baxter Fenwall, (Deerfield, IL) |

The washed material was transferred to the COBE Bone Marrow (BM) Processing Set after 10% v/v ACDA was added to the bone marrow or MPBSC.

After connecting the BM processing set to the White Blood Cell (WBC) processing set on the COBE Spectra™ device, the processor was set up using the BMP processing program, based on the hematocrit and volume of the product.

The flow rate was automatically set at 70 ml/min for products with a volume of less than 1000 ml.

The machine was set to perform a quick start, which is designed to obtain an optimal hematocrit, by automatically adjusting the plasma pump rate to remove plasma.

After quick Start was completed the plasma flow rate was manually adjusted so that the cellular material in the collection line located outside of the centrifuge was a salmon red color. This allowed the smaller lighter nucleated cells that separate on top of the red blood cells/granulocyte layer to be collected.

The marrow was processed 5-6× through the system using a two bag system connected via double coupler adapters. Clamps were placed on the lines leading from the bags so that the cells were continuously entering the machine from one bag and exiting the other. As the full bag emptied the clamps were reversed so that the cells could be processed again. The collect rate was set at 1.5 ml/min and approximately 100 ml of marrow was collected.

A cell count and flow cytometry analysis were performed on the mononuclear cell layer.

### 7.2.3. Immunomagnetic Depletion

The mononuclear cells were then incubated with an appropriate amount of anti-TCR αβ antibody (Aventis, GmBH, Marburg, Germany) in a 600 ml transfer pack at 4° C. on the rotor torque for 45 minutes. The cells were then washed on the COBE 2991™ blood cell processor at 3000 rpm using a sterile, non-pyrogenic, single-use disposable sets in a closed system. The spinning times were 2 minutes for the first three washes and 2.5 minutes for the 4^{th} wash. At the end of the run the machine was set to re-suspend the cells for 70 seconds. The cells were then incubated with sheep anti-mouse (SAM) ferromagnetic DYNABEADS (Dynal, Oslo, Norway) for 45 minutes at 4° C. on the rotor torque. At the end of the incubation a 300 ml Life cell recovery bag (Nexell Therapeutics Irvine, Calif.) was connected to the product bag containing the cells/beads. The product bag was placed on the MAXSEP® Cell Separator (Nexell Therapeutics Irvine, Calif.) and the contents were allowed to run into the recovery bag by gravity. The volume of the product was measured and samples were removed for flow cytometry analysis and cell counts.

The appropriate amount of cocktail antibodies (anti-TCR αβ, anti-CD 19, (Becton Dickinson, San Jose, Calif.), and anti-CD56 (Becton Dickinson, San Jose, Calif.)) was added to the 300 ml Life cell bag and the product was incubated for 45 minutes at 4° C. on the rotor torque.

The cells were washed on the COBE 2991™ blood cell processor at 3000 rpm using a sterile, non-pyrogenic, single-use disposable set in a closed system. The spinning times were 2 minutes for the first three washes and 2.5 minutes for the 4^{th} wash. At the end of the run the machine was set to re-suspend the cells for 70 seconds.

The cells were once again incubated with the SAM ferromagnetic DYNABEADS for 45 minutes at 4° C. on the rotor torque, at the end of which a 300 ml Life cell recovery bag was connected to the product bag containing the cells/beads. The product bag was placed on the MAXSEP® cell separator and the contents were allowed to run into the recovery bag by gravity.

The volume of the product was measured and samples were removed for flow cytometry analysis and cell counts using a FACS Calibur (Becton Dickinson Inc., San Jose, Calif.). Total nucleated cells were quantified with a Coulter ONYX (Beckman-Coulter, Miami, Fla.).

### 7.3. Results

Six small scale bone marrow preparations were processed on the COBE Spectra™ to obtain mononuclear cell layers and compared with previous methods of mononuclear cell preparation using COBE 2991™ and density gradient centrifugation, e.g., the Percoll step in Process 4 of Section 6, supra. FIG. 8 shows the comparison of CD34⁺ and lymphoid cell recoveries between the two methods. In these results, T cells are defined as αβ TCR⁺ T cells plus γδ TCR⁺ T cells.

This study indicated that use of the COBE Spectra™ processing step gave higher cell recoveries compared to previous methods. More specifically, this process step produced increases in CD34⁺, αβ TCR⁺ T cell, and 76 TCR⁺ T cell yields. The increases in doses of these cell types in the compositions of the invention are expected to improve clinical outcomes with regard to patient engraftment. Implementation of the preferred method described herein will also reduce processing time by 4-5 hours, thus reducing the time of the graft ex vivo.

### 8. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A cellular composition which facilitates donor hematopoietic progenitor cell engraftment into a recipient, comprising donor CD34⁺ hematopoietic cells in combination with donor αβ TCR⁺ T cells, wherein the CD34⁺ cells are at a concentration of about≧0.9×10⁶ cells/kg ideal body weight and the αβ TCR⁺ T cells are at a concentration of about 0.5-1×10⁵ cells/kg ideal body weight.

2. The cellular composition of claim 1 which further provides at least a 50% reduction in severe GVHD in the recipient.

3. The composition of claim 1, wherein the composition is further comprised of γδ TCR⁺ T cells.

4. The composition of claim 3, wherein the γδ TCR⁺ T cells are at a cell concentration of about≧1.5×10⁵ cells/kg ideal body weight.

5. The composition of claim 1 or 3, wherein the composition further comprises NK cells.

6. The composition of claim 1, wherein the composition comprises B cells at a dose below 0.5×10⁵ cells/kg ideal body weight.

7. A pharmaceutical composition for facilitating donor hematopoietic progenitor cell engraftment comprising the composition of claim 1, 2, 3, 4, 5 or 6.

8. A method of partially or completely reconstituting a mammalian lymphohematopoietic system comprising administering to the mammal the pharmaceutical composition of claim 7.

9. The method of claim 8 wherein the mammal is a human.

10. The method of claim 8 wherein the mammal suffers from leukemia, lymphoma or any malignancy of hematolymphoid origin.

11. A method of inducing donor-specific tolerance in a mammal in order to facilitate long term engraftment of donor cells, tissues or organs comprising administering to the mammal the pharmaceutical composition of claim 7.

12. A cellular composition which facilitates donor hematopoietic progenitor cell engraftment into a recipient, comprising donor CD34⁺ hematopoietic cells in combination with donor facilitating cells (CD8⁺/TCR⁻) and donor αβ TCR⁺ T cells, wherein the CD34⁺ cells are at a concentration of at least about 0.5×10⁶ cells/kg ideal body weight, the αβ TCR⁺ T cells are at a concentration of at least about 1×10⁴ cells/kg ideal body weight and the facilitating cells are at a concentration of at least about 0.004×10⁶ cells/kg ideal body weight.

13. The cellular composition of claim 12 which further provides at least a 50% reduction in severe GVHD in the recipient.

14. The composition of claim 12, wherein the composition is further comprised of γδ TCR⁺ T cells.

15. The composition of claim 14, wherein the γδ TCR⁺ T cells are at a cell concentration of at least about 1.5×10⁵ cells/kg ideal body weight.

16. The composition of claim 12, wherein the composition further comprises NK cells.

17. The composition of claim 1, wherein the composition comprises B cells at a dose less than about 5×10⁴ cells/kg body weight.

18. A pharmaceutical composition for facilitating donor hematopoietic progenitor cell engraftment comprising the composition of claim 12, 13, 14, 15, 16 or 17.

19. A method of partially or completely reconstituting a mammalian lymphohematopoietic system comprising administering to the mammal the pharmaceutical composition of claim 7.

20. The method of claim 19 wherein the mammal is a human.

21. The method of claim 19 wherein the mammal suffers from a disease selected from the group consisting of leukemia, lymphoma, a malignancy of hematolymphoid origin, hemoglobinopathies such as sickle cell anemia, spherocytosis, thalassemia and other blood disorders, autoimmune disease, diabetes, and metabolic disorders such as Hunters disease, Hurlers disease, and enzyme defects.

22. A method of inducing donor-specific tolerance in a mammal in order to facilitate long term engraftment of donor cells, tissues or organs comprising administering to the mammal the pharmaceutical composition of claim 18.

23. The cellular composition of claim 12 wherein the hematopoietic facilitating cells have a phenotype of CD3⁺, CD8⁺, αβ TCR⁻ and γδ TCR⁻ as determined by antibody staining and flow cytometry, which hematopoietic facilitating cells are capable of facilitating engraftment of bone marrow cells.

24. The cellular composition of claim 23 wherein the hematopoietic facilitating cells are CD45⁺.

25. The cellular composition of claim 24 wherein the hematopoietic facilitating cells are CD45R⁺.

26. The cellular composition of claim 25 wherein the hematopoietic facilitating cells are Thy1⁺, CD19− and CD56−.

27. The cellular composition of claim 26 wherein the composition comprises at least about 30% human hematopoietic facilitating cells having a phenotype of CD3⁺, CD8⁺, αβ TCR⁻ and γδ TCR⁻ as determined by antibody staining and flow cytometry.

28. The cellular composition of claim 26 wherein the composition comprises at least about 95% human hematopoietic facilitating cells having a phenotype of CD3⁺, CD8⁺, αβ TCR⁻ and γδ TCR⁻ as determined by antibody staining and flow cytometry.

29. The cellular composition of claim 12 wherein the hematopoietic facilitating cells are capable of facilitating engraftment of bone marrow cells.

30. The cellular composition of claim 29 wherein the hematopoietic facilitating cells are CD45⁺.

31. The cellular composition of claim 30 wherein the hematopoietic facilitating cells are CD45R⁺.

32. The cellular composition of claim 31 wherein the hematopoietic facilitating cells are Thy1+, CD19⁻ and CD56⁻.

33. The cellular composition of claim 29 further comprises CD34⁺ cells that are histocompatible with the hematopoietic facilitating cells.

34. The cellular composition of claim 12 wherein the hematopoietic facilitating cells are at a concentration of at least about 0.5×10⁶ cells/kg ideal body weight.

35. The cellular composition of claim 34 wherein the CD34⁺ cells are at a concentration of at least about 0.9×10⁶ cells/kg ideal body weight, the αβ TCR⁺ T cells are at a concentration of at least about 1×10⁵ cells/kg ideal body weight.

36. The cellular composition of claim 35 wherein the CD34⁺ cells are at a concentration of at least about 2×10⁶ cells/kg ideal body weight.
